(19) United States
(12) Patent Application Publication
(10) Pub. No.: US 2026/0226126 A1
(43) Pub. Date: Aug. 6, 2026

(54) ANTI-PD-1/IL-2 FUSION PROTEINS, PHARMACEUTICAL COMPOSITIONS, AND THERAPEUTIC APPLICATIONS

(71) Applicant: Anwita Biosciences, Inc., San Carlos, CA (US)

(72) Inventors: Ziyang Zhong, Belmont, CA (US); Fan Ye, Mountain View, CA (US); Jianing Huang, San Mateo, CA (US)

(73) Assignee: Anwita Biosciences, Inc., San Carlos, CA (US)

(21) Appl. No.: 19/151,374

(22) PCT Filed: Jan. 31, 2024
(86) PCT No.: PCT/US2024/013666

§ 371 Date: Jul. 28, 2025

Related U.S. Application Data
(60) Provisional application No. 63/482,790, filed on Feb. 1, 2023.

(51) Int. Cl. C07K 14/55; A61K 38/00; A61K 39/00; A61P 35/00; C07K 16/28

(52) U.S. Cl. CPC C07K 14/55; A61P 35/00; C07K 16/2818; A61K 38/00; A61K 2039/505; C07K 2317/21; C07K 2317/565; C07K 2317/569; C07K 2319/30

(57) ABSTRACT

Provided herein are anti-PD-1/IL-2 fusion proteins and pharmaceutical compositions thereof. Also provided herein are methods of their use for treating, preventing, or ameliorating one or more symptoms of a proliferative disease.

### CROSS REFERENCE TO RELATED APPLICATION

This application claims the benefit of the priority of U.S. Provisional Application No. 63/482,790, filed Feb. 1, 2023; the disclosure of which is incorporated herein by reference in its entirety.

### FIELD

Provided herein are anti-PD-1/IL-2 fusion proteins and pharmaceutical compositions thereof. Also provided herein are methods of their use for treating, preventing, or ameliorating one or more symptoms of a proliferative disease.

### REFERENCE TO A SEQUENCE LISTING

The present specification is being filed with a Sequence Listing entitled 216A020WO01_SEQLIST_ST26.XML of 238,939 bytes in size and created Jan. 31, 2024; the content of which is incorporated herein by reference in its entirety.

### BACKGROUND

Dysregulation of the host immune system is one important immune resistance mechanism for cancer. Hanahan and Weinberg, Cell 2011, 144, 646-74; Pardoll, Nat. Rev. Cancer 2012, 12, 252-64. One class of immunotherapy is agents targeting specific checkpoint proteins that play critical roles in regulating T-cell activation and proliferation. Waldman et al., Nat. Rev. Immunol. 2020, 20, 651-68. These proteins function as co-receptors on the surfaces of T-cells to help regulate T-cell responses following T-cell activation. Wolchok et al., Cancer J. 2010, 16, 311-7. The two best characterized checkpoint proteins are cytotoxic T-lymphocyte antigen 4 (CTLA-4) and programmed cell death-1 (PD-1), both serve as negative regulators of T-cell activation. Waldman et al., Nat. Rev. Immunol. 2020, 20, 651-68. T-cell activation induces expression of CTLA-4 and PD-1, thereby inhibits further T-cell activation and proliferation. Pardoll, Nat. Rev. Cancer 2012, 12, 252-64. Immune checkpoint blockade removes such inhibitory signals and unleashes antitumor immune responses. Id.; Sharma and Allison, Science 2015, 348, 56-61. Ipilimumab, a CTLA-4 blocking antibody, was the first immune checkpoint inhibitor approved by the FDA for cancer treatment. Id. Several anti-PD-1 antibodies have since been approved for cancer treatment. Gong et al., J. Immunother. Cancer 2018, 6, 8. While immunotherapy has been a major advance in cancer treatment, up to 85 percent of patients whose cancer is treated with checkpoint inhibitors do not benefit. Haslam and Prasad, JAMA Netw. Open 2019, 2, e192535.

An interleukin-2 (IL-2) is a pleiotropic cytokine that orchestrates the proliferation, survival, and function of both immune effector (Teff) cells and regulatory T (Treg) cells to maintain immune homeostasis. Bluestone, N. Engl. J. Med. 2011, 365, 2129-31; Boyman et al., Nat. Rev. Immunol. 2012, 12, 180-90. The IL-2 drives T-cell growth, augments natural killer (NK) cytolytic activity, induces the differentiation of regulatory T (Treg) cells, and mediates activation-induced cell death. Liao et al., Curr. Opin. Immunol. 2011, 23, 598-604.

An interleukin-2 receptor (IL-2R) exists in three different forms generated from three different interleukin-2 receptor chains: a chain (IL-2Rα or CD25), β chain (IL-2Rβ or CD122), and γ chain (IL-2Rγ, γ_{c}, or CD132). Wang et al., Science 2005, 310, 1159-63. The IL-2 binds the IL-2Rα with a low affinity (K_{d}≈10 nM). Id. From a crystal structure of a quaternary IL-2 signaling complex, fifteen amino acid residues (K35, T37, R38, T41, F42, K43, F44, Y45, E61, E62, K64, P65, E68, L72, and Y107) on the IL-2 are identified as interface residues between the IL-2 and IL-2Rα. Stauber et al., Proc. Natl. Acad Sci. U.S.A. 2006, 103, 2788-93. The IL-2 binds a heterodimeric complex of the IL-2Rβ and IL-2Rγ, expressed on memory T cells and NK cells, with an intermediate affinity (K_{d}≈1 nM). Wang et al., Science 2005, 310, 1159-63. The IL-2 binds a heterotrimeric complex of the IL-2Rα, IL-2Rβ, and IL-2Rγ, expressed on Treg cells, with a high affinity (K_{d}≈10 μM). Id. The IL-2 binds the IL-2Rβ alone with a dissociation constant (K_{d}) of about 100 nM and has no detectable finding affinity for the IL-2Rγ alone. Id. The IL-2Rα by itself has no signal-transducing activity. Id. The IL-2 signals through the intermediate-affinity heterodimeric IL-2Rβ/γ complex or the high-affinity heterotrimeric IL-2Rα/β/γ complex. Liao et al., Curr. Opin. Immunol. 2011, 23, 598-604. The binding of the IL-2 to the intermediate-affinity heterodimeric IL-2Rβ/γ complex leads to the activation and proliferation of immunostimulatory Teff cells, while the binding of the IL-2 to the high-affinity heterotrimeric IL-2Rα/β/γ complex results in the activation and proliferation of immunosuppressive Treg cells. Malek et al., Immunity 2010, 33, 153-65; Bluestone, N. Engl. J. Med. 2011, 365, 2129-31; Boyman et al., Nat. Rev. Immunol. 2012, 12, 180-90; Spangler et al., Annu. Rev. Immunol. 2015, 33, 139-67. This dual opposing functions of immunostimulation and immunosuppression pose a major challenge in developing the IL-2 as a safe and effective therapeutic agent. Skrombolas et al., Expert Rev. Clin. Immunol. 2014, 10, 207-17; Abbas et al., Sci. Immunol. 2018, 3, eaat1482.

Aldesleukin, a recombinant human IL-2, was approved by the FDA for metastatic renal cell carcinoma in 1992 and for metastatic melanoma in 1998. Rosenberg, J. Immunol. 2014, 192, 5451-8. Patients with metastatic melanoma or renal cancer experience a 5 to 10% rate of complete cancer regression, with an additional 10% experiencing a partial regression. Atkins et al., J. Clin. Oncol. 1999, 17, 2105-16; Klapper et al., Cancer 2008, 113, 293-301. Approximately 70% of complete responders to the IL-2 therapy do not recur. Rosenberg, Sci. Transl. Med. 2012, 4, 127ps8. However, the success of the IL-2 as an immunotherapy for cancer has been hampered by its severe toxicities and limited efficacy. One major limiting factor for its efficacy as an anticancer agent is immunosuppression resulting from the IL-2-driven preferential expansion of Treg cells. Abbas et al., Sci. Immunol. 2018, 3, eaat1482. Moreover, for the IL-2 to be effective in cancer treatment, a high dose therapeutic schedule is required. Bluestone, N. Engl. J. Med. 2011, 365, 2129-31; Abbas et al., Sci. Immunol. 2018, 3, eaat1482. This dosing regimen, however, causes vascular leak syndrome and results in the limited application of IL-2 in cancer treatment. Abbas et al., Sci. Immunol. 2018, 3, eaat1482.

Despite the advances in cancer treatment, cancer remains a major worldwide public health problem. It was estimated that there will be 1,898,160 new cancer cases diagnosed and 608,570 cancer deaths in the US alone in 2021. Cancer Facts & Figures 2021. Therefore, there is a need for an effective therapy for cancer treatment.

### SUMMARY OF THE DISCLOSURE

Provided herein is an anti-PD-1/IL-2 fusion protein comprising an IL-2 domain, an anti-PD-1 single domain antibody (sdAb), and a fragment crystallizable (Fc) domain; wherein the anti-PD-1 sdAb comprises a CDR1 of SEQ ID NO: 1, a CDR2 of SEQ ID NO: 2, and a CDR3 of SEQ ID NO: 3.

Also provided herein is an anti-PD-1/IL-2 fusion protein comprising an IL-2 domain, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the IL-2 domain directly or via a peptide linker; wherein the C-terminus of the second anti-PD-1 sdAb is connected to the N-terminus of the second peptide chain of the Fc domain directly or via a peptide linker; and wherein the anti-PD-1 sdAb comprises a CDR1 of SEQ ID NO: 1, a CDR2 of SEQ ID NO: 2, and a CDR3 of SEQ ID NO: 3.

Additionally provided herein is an anti-PD-1/IL-2 fusion protein comprising first and second IL-2 domains, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the first IL-2 domain directly or via a peptide linker; wherein the C-terminus of the second anti-PD-1 sdAb is connected to the N-terminus of the second peptide chain of the Fc domain directly or via a peptide linker, and the C-terminus of the second peptide chain of the Fc domain is connected to the N-terminus of the second IL-2 domain directly or via a peptide linker; and wherein the anti-PD-1 sdAb comprises a CDR1 of SEQ ID NO: 1, a CDR2 of SEQ ID NO: 2, and a CDR3 of SEQ ID NO: 3.

Furthermore, provided herein is a pharmaceutical composition comprising an anti-PD-1/IL-2 fusion protein provided herein, and a pharmaceutically acceptable excipient.

Provided herein is a method of treating, preventing, or ameliorating one or more symptoms of a proliferative disease in a subject, comprising administering to the subject in need thereof a therapeutically effective amount of an anti-PD-1/IL-2 fusion protein provided herein.

Provided herein is a method of inhibiting the growth of a cell, comprising contacting the cell with an effective amount of an anti-PD-1/IL-2 fusion protein provided herein.

### BRIEF DESCRIPTION OF THE DRAWINGS

FIG. 1 shows the configurations of exemplary fusion proteins: (i) an anti-PD-1/IL-2 fusion protein comprising first and second IL-2 domains, first and second anti-PD-1 sdAbs, and an Fc domain comprising first and second peptide chains; wherein the C-terminus of the first anti-PD-1 sdAb is connected to the N-terminus of the first peptide chain of the Fc domain directly, and the C-terminus of the first peptide chain of the Fc domain is connected to the N-terminus of the first IL-2 domain via a peptide linker; and wherein the C-terminus of the second anti-PD-1 sdAb is connected to the N-terminus of the second peptide chain of the Fc domain directly, and the C-terminus of the second peptide chain of the Fc domain is connected to the N-terminus of the second IL-2 domain via a peptide linker; and (ii) an anti-PD-1/IL-2 fusion protein comprising an IL-2 domain, first and second anti-PD-1 sdAbs, and an Fc domain comprising first and second peptide chains; wherein the C-terminus of the first anti-PD-1 sdAb is connected to the N-terminus of the first peptide chain of the Fc domain directly, and the C-terminus of the first peptide chain of the Fc domain is connected to the N-terminus of the IL-2 domain via a peptide linker; wherein the C-terminus of the second anti-PD-1 sdAb is connected to the N-terminus of the second peptide chain of the Fc domain directly; and wherein the Fc domain is in a knobs-into-holes configuration.

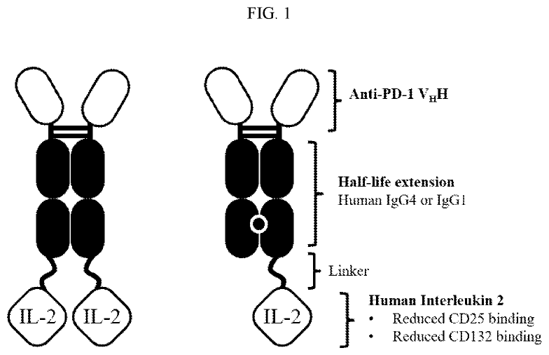

### DETAILED DESCRIPTION

To facilitate understanding of the disclosure set forth herein, a number of terms are defined below.

Generally, the nomenclature used herein and the laboratory procedures in biochemistry, biology, cell biology, immunology, molecular biology, and pharmacology described herein are those well-known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject. In one embodiment, the subject is a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

The term “therapeutically effective amount” or “effective amount” is meant to include the amount of a compound (e.g., a polypeptide or fusion protein) that, when administered, is sufficient to prevent development of, or alleviate to some extent, one or more of the symptoms of the disorder, disease, or condition being treated. The term “therapeutically effective amount” or “effective amount” also refers to the amount of a compound that is sufficient to elicit a biological or medical response of a biological molecule (e.g., a protein, enzyme, RNA, or DNA), cell, tissue, system, animal, or human, which is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of a subject (e.g., a human or an animal) without excessive toxicity, irritation, allergic response, immunogenicity, or other problems or complications, commensurate with a reasonable benefit/risk ratio. See, e.g., Remington: The Science and Practice of Pharmacy, 23rd ed.; Adejare Ed.; Academic Press, 2020; Handbook of Pharmaceutical Excipients, 9th ed.; Sheskey et al., Eds.; Pharmaceutical Press, 2020; Handbook of Pharmaceutical Additives, 3rd ed.; Ash and Ash Eds.; Synapse Information Resources, 2007; Pharmaceutical reformulation and Formulation, 1st ed.; Gibson Ed.; CRC Press, 2015.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%4%, 3%, 2%, 1%, 0.5%, or 0.05% of a given value or range.

The terms “substantially pure” and “substantially homogeneous,” when referring to a compound, mean sufficiently homogeneous to appear free of readily detectable impurities as determined by standard analytical methods used by one of ordinary skill in the art, including, but not limited to, gel electrophoresis, high performance liquid chromatography (HPLC), and mass spectrometry (MS); or sufficiently pure such that further purification would not detectably alter the physical, chemical, biological, and/or pharmacological properties, such as enzymatic and biological activities, of the compound. In certain embodiments, “substantially pure” or “substantially homogeneous” refers to a collection of molecules, wherein at least about 50%, at least about 70%, at least about 80%, at least about 90%, at least about 95%, at least about 98%, at least about 99%, or at least about 99.5% by weight of the molecules are a single compound as determined by a standard analytical method.

### Anti-PD-1/IL-2 Fusion Proteins

In one embodiment, provided herein is an anti-PD-1/IL-2 fusion protein comprising an IL-2 domain, an anti-PD-1 single domain antibody (sdAb), and a fragment crystallizable (Fc) domain; wherein the anti-PD-1 sdAb comprises a complementarity-determining region (CRD) 1 of SEQ ID NO: 1, a CDR2 of SEQ ID NO: 2, and a CDR3 of SEQ ID NO: 3.

In another embodiment, provided herein is an anti-PD-1/IL-2 fusion protein comprising one IL-2 domain, two anti-PD-1 sdAbs, and an Fc domain; wherein the anti-PD-1 sdAb comprises a CDR1 of SEQ ID NO: 1, a CDR2 of SEQ ID NO: 2, and a CDR3 of SEQ ID NO: 3.

In one embodiment, the anti-PD-1/IL-2 fusion protein provided herein comprises an IL-2 domain, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the IL-2 domain directly or via a peptide linker; and wherein the C-terminus of the second anti-PD-1 sdAb is connected to the N-terminus of the second peptide chain of the Fc domain directly or via a peptide linker.

In another embodiment, the anti-PD-1/IL-2 fusion protein provided herein comprises an IL-2 domain, first and second anti-PD-1 sdAbs, and an Fc domain comprising first and second peptide chains; wherein the C-terminus of the first anti-PD-1 sdAb is connected to the N-terminus of the first peptide chain of the Fc domain directly, and the C-terminus of the first peptide chain of the Fc domain is connected to the N-terminus of the IL-2 domain via a peptide linker; and wherein the C-terminus of the second anti-PD-1 sdAb is connected to the N-terminus of the second peptide chain of the Fc domain directly.

In yet another embodiment, provided herein is an anti-PD-1/IL-2 fusion protein comprising two IL-2 domains, two anti-PD-1 sdAbs, and an Fc domain; wherein the anti-PD-1 sdAb comprises a CDR1 of SEQ ID NO: 1, a CDR2 of SEQ ID NO: 2, and a CDR3 of SEQ ID NO: 3.

In one embodiment, the anti-PD-1/IL-2 fusion protein provided herein comprises first and second IL-2 domains, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the first IL-2 domain directly or via a peptide linker; and wherein the C-terminus of the second anti-PD-1 sdAb is connected to the N-terminus of the second peptide chain of the Fc domain directly or via a peptide linker, and the C-terminus of the second peptide chain of the Fc domain is connected to the N-terminus of the second IL-2 domain directly or via a peptide linker.

In another embodiment, the anti-PD-1/IL-2 fusion protein provided herein comprises first and second IL-2 domains, first and second anti-PD-1 sdAbs, and an Fc domain comprising first and second peptide chains; wherein the C-terminus of the first anti-PD-1 sdAb is connected to the N-terminus of the first peptide chain of the Fc domain directly, and the C-terminus of the first peptide chain of the Fc domain is connected to the N-terminus of the first IL-2 domain via a peptide linker; and wherein the C-terminus of the second anti-PD-1 sdAb is connected to the N-terminus of the second peptide chain of the Fc domain directly, and the C-terminus of the second peptide chain of the Fc domain is connected to the N-terminus of the second IL-2 domain via a peptide linker.

In one embodiment, each anti-PD-1 sdAb in an anti-PD-1/IL-2 fusion protein provided herein independently comprises the amino acid sequence of SEQ ID NO: 4 or 5. In another embodiment, each anti-PD-1 sdAb in an anti-PD-1/IL-2 fusion protein provided herein comprises the amino acid sequence of SEQ ID NO: 4. In yet another embodiment, each anti-PD-1 sdAb in an anti-PD-1/IL-2 fusion protein provided herein comprises the amino acid sequence of SEQ ID NO: 5.

In certain embodiments, the anti-PD-1 sdAb is an anti-PD-1 V_{H}H sdAb.

In one embodiment, provided herein is an anti-PD-1/IL-2 fusion protein comprising an IL-2 domain, an anti-PD-1 V_{H}H sdAb, and an Fc domain; wherein the anti-PD-1 V_{H}H sdAb comprises a CDR1 of SEQ ID NO: 1, a CDR2 of SEQ ID NO: 2, and a CDR3 of SEQ ID NO: 3.

In another embodiment, provided herein is an anti-PD-1/IL-2 fusion protein comprising one IL-2 domain, two anti-PD-1 V_{H}H sdAbs, and an Fc domain; wherein the anti-PD-1 V_{H}H sdAb comprises a CDR1 of SEQ ID NO: 1, a CDR2 of SEQ ID NO: 2, and a CDR3 of SEQ ID NO: 3.

In one embodiment, the anti-PD-1/IL-2 fusion protein provided herein comprises an IL-2 domain, first and second anti-PD-1 V_{H}H sdAbs, and an Fc domain comprising first and second peptide chains; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the IL-2 domain directly or via a peptide linker; and wherein the C-terminus of the second anti-PD-1 V_{H}H sdAb is connected to the N-terminus of the second peptide chain of the Fc domain directly or via a peptide linker.

In another embodiment, the anti-PD-1/IL-2 fusion protein provided herein comprises an IL-2 domain, first and second anti-PD-1 V_{H}H sdAbs, and an Fc domain comprising first and second peptide chains; wherein the C-terminus of the first anti-PD-1 V_{H}H sdAb is connected to the N-terminus of the first peptide chain of the Fc domain directly, and the C-terminus of the first peptide chain of the Fc domain is connected to the N-terminus of the IL-2 domain via a peptide linker; and wherein the C-terminus of the second anti-PD-1 V_{H}H sdAb is connected to the N-terminus of the second peptide chain of the Fc domain directly.

In yet another embodiment, provided herein is an anti-PD-1/IL-2 fusion protein comprising two IL-2 domains, two anti-PD-1 V_{H}H sdAbs, and an Fc domain; wherein the anti-PD-1 V_{H}H sdAb comprises a CDR1 of SEQ ID NO: 1, a CDR2 of SEQ ID NO: 2, and a CDR3 of SEQ ID NO: 3.

In one embodiment, the anti-PD-1/IL-2 fusion protein provided herein comprises first and second IL-2 domains, first and second anti-PD-1 V_{H}H sdAbs, and an Fc domain comprising first and second peptide chains; wherein the C-terminus of the first anti-PD-1 V_{H}H sdAb is connected to the N-terminus of the first peptide chain of the Fc domain directly or via a peptide linker, and the C-terminus of the first peptide chain of the Fc domain is connected to the N-terminus of the first IL-2 domain directly or via a peptide linker; and wherein the C-terminus of the second anti-PD-1 V_{H}H sdAb is connected to the N-terminus of the second peptide chain of the Fc domain directly or via a peptide linker, and the C-terminus of the second peptide chain of the Fc domain is connected to the N-terminus of the second IL-2 domain directly or via a peptide linker.

In another embodiment, the anti-PD-1/IL-2 fusion protein provided herein comprises first and second IL-2 domains, first and second anti-PD-1 V_{H}H sdAbs, and an Fc domain comprising first and second peptide chains; wherein the C-terminus of the first anti-PD-1 V_{H}H sdAb is connected to the N-terminus of the first peptide chain of the Fc domain directly, and the C-terminus of the first peptide chain of the Fc domain is connected to the N-terminus of the first IL-2 domain via a peptide linker; and wherein the C-terminus of the second anti-PD-1 V_{H}H sdAb is connected to the N-terminus of the second peptide chain of the Fc domain directly, and the C-terminus of the second peptide chain of the Fc domain is connected to the N-terminus of the second IL-2 domain via a peptide linker.

In one embodiment, each anti-PD-1 V_{H}H sdAb in an anti-PD-1/IL-2 fusion protein provided herein independently comprises the amino acid sequence of SEQ ID NO: 4 or 5. In another embodiment, each anti-PD-1 V_{H}H sdAb in an anti-PD-1/IL-2 fusion protein provided herein comprises the amino acid sequence of SEQ ID NO: 4. In yet another embodiment, each anti-PD-1 V_{H}H sdAb in an anti-PD-1/IL-2 fusion protein provided herein comprises the amino acid sequence of SEQ ID NO: 5.

In one embodiment, the Fc domain in an anti-PD-1/IL-2 fusion protein provided herein is a hIgG1 Fc domain or a mutein thereof, or a fragment thereof. In another embodiment, the Fc domain in an anti-PD-1/IL-2 fusion protein provided herein is a hIgG1 Fc having an amino acid substitution of N297A. In yet another embodiment, the Fc domain in an anti-PD-1/IL-2 fusion protein provided herein is a hIgG2 Fc domain or a mutein thereof, or a fragment thereof. In still another embodiment, the Fc domain in an anti-PD-1/IL-2 fusion protein provided herein is a hIgG4 Fc domain or a mutein thereof, or a fragment thereof.

In one embodiment, the Fc domain in an anti-PD-1/IL-2 fusion protein provided herein comprises the amino acid sequence of any one of SEQ ID NOs: 162 to 175. In yet another embodiment, the Fc domain in an anti-PD-1/IL-2 fusion protein provided herein comprises the amino acid sequence of SEQ ID NO: 162. In another embodiment, the Fc domain in an anti-PD-1/IL-2 fusion protein provided herein comprises the amino acid sequence of SEQ ID NO: 163. In yet another embodiment, the Fc domain in an anti-PD-1/IL-2 fusion protein provided herein comprises the amino acid sequence of SEQ ID NO: 164. In yet another embodiment, the Fc domain in an anti-PD-1/IL-2 fusion protein provided herein comprises the amino acid sequence of SEQ ID NO: 165. In yet another embodiment, the Fc domain in an anti-PD-1/IL-2 fusion protein provided herein comprises the amino acid sequence of SEQ ID NO: 166. In yet another embodiment, the Fc domain in an anti-PD-1/IL-2 fusion protein provided herein comprises the amino acid sequence of SEQ ID NO: 167. In yet another embodiment, the Fc domain in an anti-PD-1/IL-2 fusion protein provided herein comprises the amino acid sequence of SEQ ID NO: 168. In yet another embodiment, the Fc domain in an anti-PD-1/IL-2 fusion protein provided herein comprises the amino acid sequence of SEQ ID NO: 169. In yet another embodiment, the Fc domain in an anti-PD-1/IL-2 fusion protein provided herein comprises the amino acid sequence of SEQ ID NO: 170. In yet another embodiment, the Fc domain in an anti-PD-1/IL-2 fusion protein provided herein comprises the amino acid sequence of SEQ ID NO: 171. In yet another embodiment, the Fc domain in an anti-PD-1/IL-2 fusion protein provided herein comprises the amino acid sequence of SEQ ID NO: 172. In yet another embodiment, the Fc domain in an anti-PD-1/IL-2 fusion protein provided herein comprises the amino acid sequence of SEQ ID NO: 173. In yet another embodiment, the Fc domain in an anti-PD-1/IL-2 fusion protein provided herein comprises the amino acid sequence of SEQ ID NO: 174. In still another embodiment, the Fc domain in an anti-PD-1/IL-2 fusion protein provided herein comprises the amino acid sequence of SEQ ID NO: 175.

In one embodiment, the Fc domain in an anti-PD-1/IL-2 fusion protein provided herein comprises a pair of chains in a knobs-in-holes configuration. Thus, in one embodiment, the Fc domain in an anti-PD-1/IL-2 fusion protein provided herein comprises amino acid sequences of SEQ ID NO: 165 and 166, 167 and 168, 169 and 170, 173 and 174, or 174 and 175 as a pair of chains in a knobs-in-holes configuration. In another embodiment, the Fc domain in an anti-PD-1/IL-2 fusion protein provided herein comprises amino acid sequences of SEQ ID NO: 165 and 166 as a pair of chains in a knobs-in-holes configuration. In yet another embodiment, the Fc domain in an anti-PD-1/IL-2 fusion protein provided herein comprises amino acid sequences of SEQ ID NO: 167 and 168 as a pair of chains in a knobs-in-holes configuration. In yet another embodiment, the Fc domain in an anti-PD-1/IL-2 fusion protein provided herein comprises amino acid sequences of SEQ ID NO: 169 and 170 as a pair of chains in a knobs-in-holes configuration. In yet another embodiment, the Fc domain in an anti-PD-1/IL-2 fusion protein provided herein comprises amino acid sequences of SEQ ID NO: 173 and 174 as a pair of chains in a knobs-in-holes configuration. In still another embodiment, the Fc domain in an anti-PD-1/IL-2 fusion protein provided herein comprises amino acid sequences of SEQ ID NO: 174 and 175 as a pair of chains in a knobs-in-holes configuration.

In certain embodiments, each IL-2 domain in an anti-PD-1/IL-2 fusion protein provided herein is independently an IL-2 or a mutein thereof. In certain embodiments, each IL-2 domain in an anti-PD-1/IL-2 fusion protein provided herein is independently an IL-2 mutein.

In one embodiment, the IL-2 mutein described herein comprises, as set forth in SEQ ID NO: 46, 47, 48, 49, 50, or 51, (i) an amino acid substitution at position L18, Q126, or S130; and/or (ii) a replacement of the amino acid residues from positions N29 to L40 with a peptide comprising an amino acid sequence of an IL-15 hinge or a fragment thereof (“an IL-15 hinge fragment-containing peptide”); a disulfide bond formed between two amino acid residues from positions N30 to L80; and/or an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, 192, E95, Y107, D109, T111, or S127.

In one embodiment, the IL-2 mutein described herein comprises: (i) an amino acid substitution at position L18, Q126, or S130; and/or (ii) a replacement of the amino acid residues from positions N29 to L40 with an IL-15 hinge fragment-containing peptide; a disulfide bond formed between two amino acid residues from positions N30 to L80; an amino acid substitution at position E15, H16, D20, K32, K76, 587, N88, or I92; and/or an amino acid substitution at a position from R38 to Y45.

In another embodiment, the IL-2 mutein described herein comprises: (i) an amino acid substitution at position L18, Q126, or S130; and (ii) a replacement of the amino acid residues from positions N29 to L40 with an IL-15 hinge fragment-containing peptide; a disulfide bond formed between two amino acid residues from positions N30 to L80; or an amino acid substitution at position E15, H16, D20, K32, R38, L40, F42, K76, S87, N88, or 192.

In yet another embodiment, the IL-2 mutein described herein comprises: (i) an amino acid substitution at position L18 or Q126; and (ii) a replacement of the amino acid residues from positions N29 to L40 with an IL-15 hinge fragment-containing peptide; or an amino acid substitution at position 192.

In yet another embodiment, the IL-2 mutein described herein comprises: (i) amino acid substitutions at positions L18 and Q126; and (ii) a replacement of the amino acid residues from positions N29 to L40 with an IL-15 hinge fragment-containing peptide.

In still another embodiment, the IL-2 mutein described herein comprises: (i) an amino acid substitution at position Q126; and (ii) a replacement of the amino acid residues from positions N29 to L40 with an IL-15 hinge fragment-containing peptide; and an amino acid substitution at position 192.

In certain embodiments, the amino acid residue at position E15 is one of the twenty natural amino acids (i.e., Ala (A), Cys (C), Asp (D), Glu (E), Phe (F), Gly (G), His (H), Ile (I), Lys (K), Leu (L), Met (M), Asn (N), Pro (P), Gln (Q), Arg (R), Ser (S), Thr (T), Val (V), Trp (W), and Tyr (Y)) other than E. In certain embodiments, the amino acid residue at position E15 is K.

In certain embodiments, the amino acid residue at position H16 is one of the twenty natural amino acids other than H. In certain embodiments, the amino acid residue at position H16 is E, F, I, or V. In certain embodiments, the amino acid residue at position H16 is E, I, or V. In certain embodiments, the amino acid residue at position H16 is E. In certain embodiments, the amino acid residue at position H16 is F. In certain embodiments, the amino acid residue at position H16 is I. In certain embodiments, the amino acid residue at position H16 is V.

In certain embodiments, the amino acid residue at position L18 is one of the twenty natural amino acids other than L. In certain embodiments, the amino acid residue at position L18 is C or S. In certain embodiments, the amino acid residue at position L18 is C. In certain embodiments, the amino acid residue at position L18 is C and the amino acid residue at position C125 is C. In certain embodiments, the amino acid residue at position LIS is S.

In certain embodiments, the amino acid residue at position D20 is one of the twenty natural amino acids other than D. In certain embodiments, the amino acid residue at position D20 is A, E, K, or T. In certain embodiments, the amino acid residue at position D20 is A. In certain embodiments, the amino acid residue at position D20 is E. In certain embodiments, the amino acid residue at position D20 is K. In certain embodiments, the amino acid residue at position D20 is T.

In certain embodiments, the amino acid residue at position K32 is one of the twenty natural amino acids other than K. In certain embodiments, the amino acid residue at position K32 is D, E, or Q. In certain embodiments, the amino acid residue at position K32 is D. In certain embodiments, the amino acid residue at position K32 is E. In certain embodiments, the amino acid residue at position K32 is Q.

In certain embodiments, the amino acid residue at position R38 is one of the twenty natural amino acids other than R. In certain embodiments, the amino acid residue at position R38 is E or N. In certain embodiments, the amino acid residue at position R38 is E. In certain embodiments, the amino acid residue at position R38 is N.

In certain embodiments, the amino acid residue at position L40 is one of the twenty natural amino acids other than L. In certain embodiments, the amino acid residue at position L40 is S or T. In certain embodiments, the amino acid residue at position L40 is S. In certain embodiments, the amino acid residue at position L40 is T.

In certain embodiments, the amino acid residue at position F42 is one of the twenty natural amino acids other than F. In certain embodiments, the amino acid residue at position F42 is A, C, K, or N. In certain embodiments, the amino acid residue at position F42 is A or K. In certain embodiments, the amino acid residue at position F42 is A. In certain embodiments, the amino acid residue at position F42 is C. In certain embodiments, the amino acid residue at position F42 is K. In certain embodiments, the amino acid residue at position F42 is N.

In certain embodiments, the amino acid residue at position K76 is one of the twenty natural amino acids other than K. In certain embodiments, the amino acid residue at position K76 is D, E, or Q. In certain embodiments, the amino acid residue at position K76 is D. In certain embodiments, the amino acid residue at position K76 is E. In certain embodiments, the amino acid residue at position K76 is Q.

In certain embodiments, the amino acid residue at position S87 is one of the twenty natural amino acids other than S. In certain embodiments, the amino acid residue at position S87 is D or E. In certain embodiments, the amino acid residue at position S87 is D. In certain embodiments, the amino acid residue at position S87 is E.

In certain embodiments, the amino acid residue at position N88 is one of the twenty natural amino acids other than N. In certain embodiments, the amino acid residue at position N88 is A.

In certain embodiments, the amino acid residue at position 192 is one of the twenty natural amino acids other than I. In certain embodiments, the amino acid residue at position 192 is A, D, E, or G. In certain embodiments, the amino acid residue at position 192 is A. In certain embodiments, the amino acid residue at position 192 is D. In certain embodiments, the amino acid residue at position 192 is E. In certain embodiments, the amino acid residue at position 192 is G.

In certain embodiments, the amino acid residue at position Q126 is one of the twenty natural amino acids other than Q. In certain embodiments, the amino acid residue at position Q126 is E, K, R, S, or T. In certain embodiments, the amino acid residue at position Q126 is E, K, or R. In certain embodiments, the amino acid residue at position Q126 is E. In certain embodiments, the amino acid residue at position Q126 is K. In certain embodiments, the amino acid residue at position Q126 is R. In certain embodiments, the amino acid residue at position Q126 is S. In certain embodiments, the amino acid residue at position Q126 is T.

In certain embodiments, the amino acid residue at position S130 is one of the twenty natural amino acids other than S. In certain embodiments, the amino acid residue at position S130 is A, E, Q, or R. In certain embodiments, the amino acid residue at position S130 is A. In certain embodiments, the amino acid residue at position S130 is E. In certain embodiments, the amino acid residue at position S130 is Q. In certain embodiments, the amino acid residue at position S130 is R.

In one embodiment, the IL-2 mutein described herein comprises: (i) an amino acid substitution of L18C, L18S, Q126E, Q126K, Q126R, Q126S, Q126T, S130A, S130E, S130Q, or S130R; and/or (ii) a replacement of the amino acid residues from positions N29 to L40 having the amino acid sequence of SEQ ID NO: 84 with an IL-15 hinge fragment-containing peptide; a disulfide bond formed between two different amino acid residues, each independently at position K35, R38, F42, Y45, E62, V69, or L72; and/or an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In another embodiment, the IL-2 mutein described herein comprises: (i) an amino acid substitution of L18C, Q126E, Q126K, Q126R, Q126S, Q126T, S130A, S130E, S130Q, or S130R; and/or (ii) a replacement of the amino acid residues from positions N29 to L40 having the amino acid sequence of SEQ ID NO: 84 with an IL-15 hinge fragment-containing peptide; a disulfide bond formed between two different amino acid residues, each independently at position K35, R38, F42, Y45, E62, V69, or L72; and/or an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, L18S, Q126E, Q126K, Q126R, S130A, S130E, S130Q, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, Q126E, Q126K, Q126R, S130A, S130E, S130Q, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, L18S, Q126E, Q126K, Q126R, S130A, S130E, S130Q, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, Q126E, Q126K, or S130R; and (ii) a replacement of the amino acid residues from positions N29 to L40 having the amino acid sequence of SEQ ID NO: 84 with an IL-15 hinge fragment-containing peptide; a disulfide bond formed between F42C and V69C; or an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18S or Q126E; and (ii) a replacement of the amino acid residues from positions N29 to L40 having the amino acid sequence of SEQ ID NO: 84 with an IL-15 hinge fragment-containing peptide; or an amino acid substitution of I92A.

In yet another embodiment, the IL-2 mutein described herein comprises: (i) amino acid substitutions of L18S and Q126E; and (ii) a replacement of the amino acid residues from positions N29 to L40 having the amino acid sequence of SEQ ID NO: 84 with an IL-15 hinge fragment-containing peptide.

In still another embodiment, the IL-2 mutein described herein comprises: (i) an amino acid substitution of Q126E; and (ii) a replacement of the amino acid residues from positions N29 to L40 having the amino acid sequence of SEQ ID NO: 84 with an IL-15 hinge fragment-containing peptide; and an amino acid substitution of I92A.

In one embodiment, the IL-15 hinge fragment-containing peptide comprises the amino acid sequence of SEQ ID NO: 85 or 86. In another embodiment, the IL-15 hinge fragment-containing peptide comprises the amino acid sequence of SEQ ID NO: 85. In yet another embodiment, the IL-15 hinge fragment-containing peptide comprises the amino acid sequence of SEQ ID NO: 86.

In one embodiment, the IL-2 mutein described herein comprises: (i) an amino acid substitution of L18C, L18S, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In another embodiment, the IL-2 mutein described herein comprises: (i) an amino acid substitution of L18C, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, L18S, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, L18S, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 86; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, Q126E, Q126K, or S130R; and (ii) a replacement of the amino acid residues from positions N29 to L40 having the amino acid sequence of SEQ ID NO: 84 with an IL-15 hinge fragment-containing peptide having the amino acid sequence of SEQ ID NO: 86; a disulfide bond formed between F42C and V69C; or an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18S or Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85; or an amino acid substitution of I92A.

In yet another embodiment, the IL-2 mutein described herein comprises: (i) an amino acid substitution of L18S or Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 86; or an amino acid substitution of I92A.

In yet another embodiment, the IL-2 mutein described herein comprises: (i) amino acid substitutions of L18S and Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85.

In yet another embodiment, the IL-2 mutein described herein comprises: (i) amino acid substitutions of L18S and Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 86.

In yet another embodiment, the IL-2 mutein described herein comprises: (i) an amino acid substitution of Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 85; and an amino acid substitution of I92A.

In still another embodiment, the IL-2 mutein described herein comprises: (i) an amino acid substitution of Q126E; and (ii) a replacement of the amino acid residues from positions N29 to L40 having the amino acid sequence of SEQ ID NO: 84 with an IL-15 hinge fragment-containing peptide having the amino acid sequence of SEQ ID NO: 86; and an amino acid substitution of I92A.

In one embodiment, the IL-2 mutein described herein comprises: (i) an amino acid substitution at position L18, Q126, or S130; and (ii) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, S87, N88, V91, 192, E95, Y107, D109, T111, or S127.

In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69C, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein described herein comprises: (i) an amino acid substitution at position L18, Q126, or S130; and (ii) an amino acid substitution at position E15, H16, D20, K32, R38, L40, F42, K76, S87, N88, or 192.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of E15K, H16E, H16F, H16I, H16V, D20A, D20E, D20K, D20T, K32E, R38E, R38N, L40S, L40T, F42A, F42K, F42N, K76D, K76E, K76Q, S87D, S87E, N88A, I92A, I92D, I92E, or I92G.

In yet another embodiment, the IL-2 mutein described herein comprises: (i) an amino acid substitution of L18C, L18S, Q126E, Q126K, Q126R, S130A, S130E, S130Q, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, Q126E, Q126K, Q126R, S130A, S130E, S130Q, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In yet another embodiment, the IL-2 mutein described herein comprises: (i) an amino acid substitution of L18C, L18S, Q126E, Q126K, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In still another embodiment, the IL-2 mutein described herein comprises: (i) an amino acid substitution of L18C, Q126E, Q126K, or S130R; and (ii) an amino acid substitution of E15K, H16E, H16F, H16I, H16V, D20A, D20E, D20K, D20T, K32E, R38E, R38N, L40T, F42A, F42K, K76E, S87D, N88A, I92A, I92D, I92E, or I92G.

In one embodiment, the IL-2 mutein described herein comprises an amino acid substitution at position L18. In another embodiment, the IL-2 mutein described herein comprises an amino acid substitution of L18C or L18S. In yet another embodiment, the IL-2 mutein described herein comprises an amino acid substitution of L18C. In still another embodiment, the IL-2 mutein described herein comprises an amino acid substitution of L18S.

In one embodiment, the IL-2 mutein described herein described herein comprises an amino acid substitution at position Q126. In another embodiment, the IL-2 mutein described herein comprises an amino acid substitution of Q126E, Q126K, Q126R, Q126S, or Q126T. In yet another embodiment, the IL-2 mutein described herein comprises an amino acid substitution of Q126E. In yet another embodiment, the IL-2 mutein described herein described herein comprises an amino acid substitution of Q126K. In yet another embodiment, the IL-2 mutein described herein comprises an amino acid substitution of Q126R. In yet another embodiment, the IL-2 mutein described herein comprises an amino acid substitution of Q126S. In still another embodiment, the IL-2 mutein described herein comprises an amino acid substitution of Q126T.

In one embodiment, the IL-2 mutein described herein comprises an amino acid substitution at position S130. In another embodiment, the IL-2 mutein described herein comprises an amino acid substitution of S130A, S130E, S130Q, or S130R. In yet another embodiment, the IL-2 mutein described herein comprises an amino acid substitution of S130A. In yet another embodiment, the IL-2 mutein described herein comprises an amino acid substitution of S130E. In yet another embodiment, the IL-2 mutein described herein comprises an amino acid substitution of S130Q. In still another embodiment, the IL-2 mutein described herein comprises an amino acid substitution of S130R.

In one embodiment, the IL-2 mutein described herein comprises amino acid substitutions at positions L18 and S126. In another embodiment, the IL-2 mutein described herein comprises an amino acid substitution of L18C or L18S and an amino acid substitution of Q126E, Q126K, Q126R, Q126S, or Q126T. In yet another embodiment, the IL-2 mutein described herein comprises an amino acid substitution of L18C and an amino acid substitution of Q126E, Q126K, Q126R, Q126S, or Q126T. In yet another embodiment, the IL-2 mutein described herein comprises amino acid substitutions of L18C and Q126E. In yet another embodiment, the IL-2 mutein described herein comprises amino acid substitutions of L18C and Q126K. In yet another embodiment, the IL-2 mutein described herein comprises amino acid substitutions of L18C and Q126R. In yet another embodiment, the IL-2 mutein described herein comprises amino acid substitutions of L18C and Q126S. In still another embodiment, the IL-2 mutein described herein comprises amino acid substitutions of L18C and Q126T.

In one embodiment, the IL-2 mutein described herein comprises an amino acid substitution of L18S and an amino acid substitution of Q126E, Q126K, Q126R, Q126S, or Q126T. In another embodiment, the IL-2 mutein described herein comprises amino acid substitutions of L18S and Q126E. In yet another embodiment, the IL-2 mutein described herein comprises amino acid substitutions of L18S and Q126K. In yet another embodiment, the IL-2 mutein described herein comprises amino acid substitutions of L18S and Q126R. In yet another embodiment, the IL-2 mutein described herein comprises amino acid substitutions of L18S and Q126S. In still another embodiment, the IL-2 mutein described herein comprises amino acid substitutions of L18S and Q126T.

In one embodiment, the IL-2 mutein described herein comprises amino acid substitutions at positions S126 and S130. In another embodiment, the IL-2 mutein described herein comprises an amino acid substitution of Q126E, Q126K, Q126R, Q126S, or Q126T, and an amino acid substitution of S130A, S130E, S130Q, or S130R. In yet another embodiment, the IL-2 mutein described herein comprises an amino acid substitution of Q126E, Q126K, Q126R, Q126S, or Q126T, and an amino acid substitution of S130E or S130R.

In one embodiment, the IL-2 mutein described herein comprises: (i) an amino acid substitution at position L18, Q126, or S130; and (ii) an amino acid substitution at position E15. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of E15K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of E15K.

In one embodiment, the IL-2 mutein described herein comprises: (i) an amino acid substitution at position L18, Q126, or S130; and (ii) an amino acid substitution at position H16. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of H16E, H16F, H16I, or H16V. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16E, H16F, H16I, or H16V. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16E, H16I, or H16V. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16F. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16I. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of H16V.

In one embodiment, the IL-2 mutein described herein comprises: (i) an amino acid substitution at position L18, Q126, or S130; and (ii) an amino acid substitution at position D20. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of D20A, D20E, D20K, or D20T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of D20A, D20E, D20K, or D20T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of D20A. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of D20E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of D20K. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of D20T.

In one embodiment, the IL-2 mutein described herein comprises: (i) an amino acid substitution at position L18, Q126, or S130; and (ii) an amino acid substitution at position K32. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of K32E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of K32E.

In one embodiment, the IL-2 mutein described herein comprises: (i) an amino acid substitution at position L18, Q126, or S130; and (ii) an amino acid substitution at position R38. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of R38E or R38N. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of R38E or R38N. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of R38E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of R38N.

In one embodiment, the IL-2 mutein described herein comprises: (i) an amino acid substitution at position L18, Q126, or S130; and (ii) an amino acid substitution at position L40. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of L40S or L40T. In yet another embodiment, the IL-2 mutein described herein comprises: (i) an amino acid substitution of L18C, Q126E, Q126K, Q126R, Q126S, Q126T, S130A, S130E, S130Q, or S130R, and (ii) an amino acid substitution of L40S or L40T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of L40S. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of L40T.

In one embodiment, the IL-2 mutein described herein comprises: (i) an amino acid substitution at position L18, Q126, or S130; and (ii) an amino acid substitution at position F42. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of F42A, F42C, or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of F42A, F42C, or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of F42A. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of F42C. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of F42K.

In one embodiment, the IL-2 mutein described herein comprises: (i) an amino acid substitution at position L18, Q126, or S130; and (ii) an amino acid substitution at position K76. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of K76E.

In one embodiment, the IL-2 mutein described herein comprises: (i) an amino acid substitution at position L18, Q126, or S130; and (ii) an amino acid substitution at position S87. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of S87D. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of S87D.

In one embodiment, the IL-2 mutein described herein comprises: (i) an amino acid substitution at position L18, Q126, or S130; and (ii) an amino acid substitution at position N88. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of N88A. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of N88A.

In one embodiment, the IL-2 mutein described herein comprises: (i) an amino acid substitution at position L18, Q126, or S130; and (ii) an amino acid substitution at position I92. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) an amino acid substitution of I92A, I92D, I92E, or I92G. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of I92A, I92D, I92E, or I92G. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of I92A. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of I92D. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of I92E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) an amino acid substitution of I92G.

In one embodiment, the IL-2 mutein described herein comprises amino acid substitutions at positions 192 and S126. In another embodiment, the IL-2 mutein described herein comprises an amino acid substitution of I92A, I92D, I92E, or I92G, and an amino acid substitution of Q126E, Q126K, Q126R, Q126S, or Q126T. In yet another embodiment, the IL-2 mutein described herein comprises an amino acid substitution of I92A or I92G, and an amino acid substitution of Q126E, Q126K, Q126R, Q126S, or Q126T. In yet another embodiment, the IL-2 mutein described herein comprises amino acid substitutions of I92A and Q126E. In yet another embodiment, the IL-2 mutein described herein comprises amino acid substitutions of I92A and Q126K. In yet another embodiment, the IL-2 mutein described herein comprises amino acid substitutions of I92A and Q126R. In yet another embodiment, the IL-2 mutein described herein comprises amino acid substitutions of I92A and Q126S. In still another embodiment, the IL-2 mutein described herein comprises amino acid substitutions of I92A and Q126T.

In one embodiment, the IL-2 mutein described herein comprises: (i) an amino acid substitution at position L18, Q126, or S130; and (ii) amino acid substitutions at positions R38 and L40. In another embodiment, the IL-2 mutein described herein comprises: (i) an amino acid substitution at position L18, Q126, or S130; and (ii) amino acid substitutions at positions E15, R38, and L40. In yet another embodiment, the IL-2 mutein described herein comprises: (i) an amino acid substitution at position L18, Q126, or S130; and (ii) amino acid substitutions at positions R38, L40, and K76. In still another embodiment, the IL-2 mutein described herein comprises: (i) an amino acid substitution at position L18, Q126, or S130; and (ii) amino acid substitutions at positions E15, R38, L40, and K76.

In one embodiment, the IL-2 mutein described herein comprises: (i) an amino acid substitution at position L18, Q126, or S130; and (ii) amino acid substitutions of R38N and L40S or L40T. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40S or L40T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N and L40S or L40T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38N, L40S or L40T, and K76E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N, L40S or L40T, and K76E.

In one embodiment, the IL-2 mutein described herein comprises: (i) an amino acid substitution at position L18, Q126, or S130; and (ii) amino acid substitutions of R38N and L40S. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40S. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N and L40S. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38N, L40S, and K76E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N, L40S, and K76E.

In one embodiment, the IL-2 mutein described herein comprises: (i) an amino acid substitution at position L18, Q126, or S130; and (ii) amino acid substitutions of R38N and L40T. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of E15K, R38N and L40T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N and L40T. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38N, L40T, and K76E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38N, L40T, and K76E.

In one embodiment, the IL-2 mutein described herein is glycosylated. In another embodiment, the IL-2 mutein described herein is N-glycosylated. In yet another embodiment, the IL-2 mutein described herein is glycosylate at the nitrogen in the side chain of an asparagine residue. In still another embodiment, the IL-2 mutein described herein comprises: (i) an amino acid substitution at position L18, Q126, or S130; and (ii) an amino acid substitution of R38N that is N-glycosylated.

In one embodiment, the IL-2 mutein described herein is an N-glycosylated IL-2 mutein comprising: (i) an amino acid substitution at position L18, Q126, or S130; and (ii) amino acid substitutions at positions R38 and L40. In another embodiment, the IL-2 mutein described herein is an N-glycosylated IL-2 mutein comprising: (i) an amino acid substitution at position L18, Q126, or S130; (ii) amino acid substitutions at positions E15, R38, and L40; and (iii) optionally an amino acid substitution at position H16, D20, K76, S87, N88, or I92. In yet another embodiment, the IL-2 mutein described herein is an N-glycosylated IL-2 mutein comprising: (i) an amino acid substitution of at position L18, Q126, or S130; (ii) amino acid substitutions at positions R38, L40, and K76; and (iii) optionally an amino acid substitution at position E15, H16, D20, S87, N88, or I92. In still another embodiment, the IL-2 mutein described herein is an N-glycosylated IL-2 mutein comprising: (i) an amino acid substitution of at position L18, Q126, or S130; (ii) amino acid substitutions at positions E15, R38, L40, and K76; and (iii) optionally an amino acid substitution at position H16, D20, S87, N88, or 192.

In one embodiment, the IL-2 mutein described herein is an N-glycosylated IL-2 mutein comprising: (i) an amino acid substitution at position LIS, Q126, or S130; and (ii) amino acid substitutions of R38N and L40S or L40T. In another embodiment, the IL-2 mutein described herein is an N-glycosylated IL-2 mutein: (i) an amino acid substitution of L18C, L18S, Q126E, Q126K, Q126R, Q126S, Q126T, S130A, S130E, S130Q, or S130R; and (ii) comprising amino acid substitutions of E15K, R38N and L40S or L40T. In yet another embodiment, the IL-2 mutein described herein is an N-glycosylated IL-2 mutein: (i) an amino acid substitution of L18C, Q126E, Q126K, Q126R, Q126S, Q126T, S130A, S130E, S130Q, or S130R; and (ii) comprising amino acid substitutions of E15K, R38N and L40S or L40T. In yet another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40S or L40T, and K76E. In still another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N, L40S or L40T, and K76E.

In one embodiment, the IL-2 mutein described herein is an N-glycosylated IL-2 mutein comprising: (i) an amino acid substitution at position L18, Q126, or S130; and (ii) amino acid substitutions of R38N and L40S. In another embodiment, the IL-2 mutein described herein is an N-glycosylated IL-2 mutein comprising: (i) an amino acid substitution of L18C, L18S, Q126E, Q126K, Q126R, Q126S, Q126T, S130A, S130E, S130Q, or S130R; and (ii) amino acid substitutions of E15K, R38N and L40S. In yet another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N and L40S. In yet another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40S, and K76E. In still another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R, and (ii) amino acid substitutions of E15K, R38N, L40S, and K76E.

In one embodiment, the IL-2 mutein described herein is an N-glycosylated IL-2 mutein comprising: (i) an amino acid substitution at position L18, Q126, or S130; and (ii) amino acid substitutions of R38N and L40T. In another embodiment, the IL-2 mutein described herein is an N-glycosylated IL-2 mutein comprising: (i) an amino acid substitution of L18C, L18S, Q126E, Q126K, Q126R, Q126S, Q126T, S130A, S130E, S130Q, or S130R; and (ii) amino acid substitutions of E15K, R38N and L40T. In yet another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N and L40T. In yet another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of R38N, L40T, and K76E. In still another embodiment, the IL-2 mutein described herein is an N-glycosylated IL-2 mutein comprising: (i) an amino acid substitution of L18C, Q126E, Q126K, Q126R, Q126S, Q126T, S130A, S130E, S130Q, or S130R; and (ii) amino acid substitutions of E15K, R38N, L40T, and K76E.

In one embodiment, the N-glycosylated IL-2 mutein provided herein comprises one glycan. In another embodiment, the N-glycosylated IL-2 mutein provided herein comprises one glycan attached to the nitrogen in the side chain of an asparagine residue. In yet another embodiment, the N-glycosylated IL-2 mutein provided herein comprises one glycan attached to the nitrogen in the side chain of an asparagine residue at position R38N.

In one embodiment, the N-glycosylated IL-2 mutein provided herein comprises two glycans. In another embodiment, the N-glycosylated IL-2 mutein provided herein comprises two glycans, of which at least one glycan is attached to the nitrogen in the side chain of an asparagine residue. In yet another embodiment, the N-glycosylated IL-2 mutein provided herein comprises two glycans, each of which is attached to the nitrogen in the side chain of an asparagine residue.

In one embodiment, the N-glycosylated IL-2 mutein provided herein comprises three glycans. In one embodiment, the glycan is an N-glycan.

In one embodiment, the N-glycan on the N-glycosylated IL-2 mutein provided herein is oligomannose-type. In another embodiment, the N-glycan on the N-glycosylated IL-2 mutein provided herein is complex-type. In another embodiment, the N-glycan on the N-glycosylated IL-2 mutein provided herein is hydride-type.

In one embodiment, the N-glycan on the N-glycosylated IL-2 mutein provided herein is biantennary complex-type. In another embodiment, the N-glycan on the N-glycosylated IL-2 mutein provided herein is triantennary complex-type. In yet another embodiment, the N-glycan on the N-glycosylated IL-2 mutein provided herein is tetraantennary complex-type.

In one embodiment, the N-glycan on the N-glycosylated IL-2 mutein provided herein is one of the glycans described in Szabo et al., J. Proteome. Res. 2018, 17, 1559-1574, the disclosure of which is incorporated herein by reference in its entirety.

In one embodiment, the IL-2 mutein described herein comprises: (i) an amino acid substitution at position L18, Q126, or S130; and (ii) amino acid substitutions at positions R38 and F42. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions at positions K32, R38, and F42. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions at positions K32, R38, and F42. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions at positions R38, F42, and K76. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions at positions E15, K32, R38, and F42. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions at positions E15, R38, F42, and K76.

In one embodiment, the IL-2 mutein described herein comprises: (i) an amino acid substitution at position L18, Q126, or S130; and (ii) amino acid substitutions of R38E and F42A or F42K. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A or F42K. In still another embodiment, the IL-2 mutein described herein comprises: (i) an amino acid substitution of L18C, Q126E, Q126K, Q126R, Q126S, Q126T, S130A, S130E, S130Q, or S130R, and (ii) amino acid substitutions of E15K, R38E, F42A or F42K, and K76E.

In one embodiment, the IL-2 mutein described herein comprises: (i) an amino acid substitution at position L18, Q126, or S130; and (ii) amino acid substitutions at positions K32, R38, and F42. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42A. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of K32E, R38E, and F42K.

In one embodiment, the IL-2 mutein described herein comprises: (i) an amino acid substitution at position L18, Q126, or S130; and (ii) amino acid substitutions at positions R38, F42, and K76. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38E, F42A or F42K, and K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38E, F42A, and K76E. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of R38E, F42K, and K76E.

In one embodiment, the IL-2 mutein described herein comprises: (i) an amino acid substitution at position L18, Q126, or S130; and (ii) amino acid substitutions at positions E15, K32, R38, and F42. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of E15K, K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A or F42K. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42A. In still another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, K32E, R38E, and F42K.

In one embodiment, the IL-2 mutein described herein comprises: (i) an amino acid substitution at position L18, Q126, or S130; and (ii) amino acid substitutions at positions E15, R38, F42, and K76. In another embodiment, the IL-2 mutein described herein comprises: (i) an amino acid substitution of L18C, L18S, Q126E, Q126K, Q126R, Q126S, Q126T, S130A, S130E, S130Q, or S130R; and (ii) amino acid substitutions of E15K, R38E, F42A or F42K, and K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38E, F42A or F42K, and K76E. In yet another embodiment, the IL-2 mutein described herein comprises: (i) an amino acid substitution of L18C, Q126E, Q126K, Q126R, Q126S, Q126T, S130A, S130E, S130Q, or S130R; and (ii) amino acid substitutions of E15K, R38E, F42A, and K76E. In still another embodiment, the IL-2 mutein described herein comprises: (i) an amino acid substitution of L18C, Q126E, Q126K, Q126R, Q126S, Q126T, S130A, S130E, S130Q, or S130R; and (b) amino acid substitutions of E15K, R38E, F42K, and K76E.

In one embodiment, the IL-2 mutein described herein comprises: (i) an amino acid substitution at position L18, Q126, or S130; and (ii) a disulfide bond formed between two different amino acid residues from positions N30 to L80. In another embodiment, the IL-2 mutein described herein comprises: (i) an amino acid substitution at position L18, Q126, or S130; and (ii) a disulfide bond formed between two different amino acid residues from positions K35 to L72. In yet another embodiment, the IL-2 mutein described herein comprises: (i) an amino acid substitution at position L18, Q126, or S130; and (ii) a disulfide bond formed between two different amino acid residues from positions K35 to L69.

In one embodiment, the IL-2 mutein described herein comprises: (i) an amino acid substitution at position L18, Q126, or S130; and (ii) a disulfide bond formed between two different amino acid residues, each independently at K35, R38, F42, Y45, E62, V69, or L72. In another embodiment, the IL-2 mutein described herein comprises: (i) an amino acid substitution at position L18, Q126, or S130; and (ii) a disulfide bond formed between an amino acid residue at position K35, R38, F42, or Y45; and an amino acid residue at position E62, V69, or L72. In yet another embodiment, the IL-2 mutein described herein comprises: (i) an amino acid substitution at position L18, Q126, or S130; and (ii) a disulfide bond formed between an amino acid residue at position F42 and an amino acid residue at position E62, V69, or L72. In still another embodiment, the IL-2 mutein described herein comprises: (i) an amino acid substitution at position L18, Q126, or S130; and (ii) a disulfide bond formed between an amino acid residue at position K35, R38, F42, or Y45, and an amino acid residue at position V69.

In one embodiment, the IL-2 mutein described herein comprises: (i) an amino acid substitution of L18C, L18S, Q126E, Q126K, Q126R, Q126S, Q126T, S130A, S130E, S130Q, or S130R; and (ii) a disulfide bond formed between K35C and L72C, R38C and L72C, F42C and V69C, Y42C and L72C, or Y45C and E62C. In another embodiment, the IL-2 mutein described herein comprises: (i) an amino acid substitution of L18C, Q126E, Q126K, Q126R, Q126S, Q126T, S130A, S130E, S130Q, or S130R; and (ii) a disulfide bond formed between K35C and L72C, R38C and L72C, F42C and V69C, Y42C and L72C, or Y45C and E62C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between K35C and L72C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between R38C and L72C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between F42C and V69C. In yet another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between Y42C and L72C. In still another embodiment, the IL-2 domain in the fusion protein comprises: (i) an amino acid substitution of L18C, Q126E, Q126K, Q126R, Q126S, Q126T, S130A, S130E, S130Q, or S130R; and (ii) a disulfide bond formed between Y45C and E62C.

In one embodiment, the IL-2 mutein described herein comprises: (i) an amino acid substitution at position L18, Q126, or S130; and (ii) a disulfide bond formed between two different amino acid residues from positions N30 to L80; and (iii) an amino acid substitution at position K8, K9, Q13, E15, H16, L19, D20, M23, K32, P34, K35, L36, T37, R38, M39, L40, T41, F42, K43, F44, Y45, M46, P47, E61, E62, L63, K64, P65, L66, E67, E68, V69, L70, N71, L72, A73, Q74, K76, H79, R81, D84, 587, N88, V91, 192, E95, Y107, D109, T1, or 5127.

In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a disulfide bond formed between two different amino acid residues, each independently at position K35, R38, F42, Y45, E62, V69, or L72; and (iii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, E95K, E95N, E95Q, Y107N, D109N, T111S, S127A, S127E, S127F, or S127W.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between two different amino acid residues, each independently at position K35, R38, F42, Y45, E62, V69, or L72; and (iii) an amino acid substitution of K8D, K8E, K8Q, K9D, K9E, K9Q, Q13E, Q13N, E15K, E15Q, E15V, H16E, H16F, H16I, H16V, L19S, D20A, D20E, D20K, D20T, M23K, K32D, K32E, K32Q, P34N, K35N, L36S, L36T, T37N, R38E, R38N, M39N, L40S, L40T, T41N, F42A, F42C, F42K, F42N, K43N, F44N, Y45N, M46S, M46T, P47S, P47T, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, E95K, E95N, E95Q, Y107N, D109N, T111S, S127A, S127E, S127F, or S127W.

In yet another embodiment, the IL-2 mutein described herein comprises (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16F, H16I, H16V, D20A, D20E, D20K, D20T, K32E, K76E, S87D, N88A, I92A, I92D, I92E, or I92G.

In yet another embodiment, the IL-2 mutein described herein comprises (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16F, H16I, H16V, D20A, D20E, D20K, D20T, K32E, K76E, S87D, N88A, I92A, I92D, I92E, or I92G.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16I, H16V, D20A, D20E, D20K, D20T, K32E, K76E, S87D, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, H16E, H16I, H16V, D20A, D20E, D20K, D20T, K32E, K76E, S87D, N88A, I92A, I92D, I92E, or I92G.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a disulfide bond formed between F42C and V69C; and (iii) an amino acid substitution of E15K, K32E, or K76E.

In still another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of E15K, K32E, or K76E.

In one embodiment, the IL-2 mutein described herein comprises: (i) an amino acid substitution at position L18, Q126, or S130; and (ii) a disulfide bond formed between F42C and V69C. In another embodiment, the IL-2 mutein described herein comprises: (i) an amino acid substitution at position L18, Q126, or S130; (ii) a disulfide bond formed between F42C and V69C; an amino acid substitution at position K32 or K76; and (iii) optionally an amino acid substitution at position E15. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of K32E. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) an amino acid substitution of K76E. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) amino acid substitutions of E15K and K32E. In still another embodiment, the IL-2 mutein described herein comprises: (i) an amino acid substitution of L18C, Q126E, Q126K, Q126R, Q126S, Q126T, S130A, S130E, S130Q, or S130R; (ii) a disulfide bond formed between F42C and V69C; and (iii) amino acid substitutions of E15K and K76E.

In one embodiment, the IL-2 mutein described herein comprises: (i) an amino acid substitution at position L18, Q126, or S130; (ii) a replacement of the amino acid residues from positions N29 to L40 with an IL-15 hinge fragment-containing peptide. In another embodiment, the IL-2 mutein described herein comprises a replacement of the amino acid residues from positions N29 to L40 having the amino acid sequence of SEQ ID NO: 84 with an IL-15 hinge fragment-containing peptide having the amino acid sequence of SEQ ID NO: 85 or 86. In yet another embodiment, the IL-2 mutein described herein comprises a replacement of the amino acid residues from positions N29 to L40 having the amino acid sequence of SEQ ID NO: 84 with an IL-15 hinge fragment-containing peptide having the amino acid sequence of SEQ ID NO: 85. In still another embodiment, the IL-2 mutein described herein comprises a replacement of the amino acid residues from positions N29 to L40 having the amino acid sequence of SEQ ID NO: 84 with an IL-15 hinge fragment-containing peptide having the amino acid sequence of SEQ ID NO: 86.

In one embodiment, the IL-2 mutein described herein comprises: (i) an amino acid substitution at position L18, Q126, or S130; (ii) a replacement of the amino acid residues from positions N29 to L40 with an IL-15 hinge fragment-containing peptide; and (iii) an amino acid substitution at position K8, K9, Q13, E15, H16, L19, D20, M23, E61, E62, L63, K64, P65, L66, E67, E68, V69, L70, N71, L72, A73, Q74, K76, H79, R81, D84, S87, N88, V91, 192, E95, Y107, D109, T111, or S127.

In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73 T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In still another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of K8D, K8E, K8Q, K9D, K9E, K9Q, Q13E, Q13N, E15K, E15Q, E15V, H16D, H16E, H16F, H16I, H16N, H16Q, H16V, L19S, D20A, D20E, D20K, D20T, M23K, E61N, E62N, L63S, L63T, K64N, P65N, L66N, E67S, E67T, E68N, V69N, L70S, L70T, N71S, N71T, L72N, A73S, A73 T, Q74S, Q74T, K76D, K76E, K76Q, H79D, H79E, H79Q, R81D, R81E, R81Q, D84T, S87D, S87E, N88A, V91I, I92A, I92D, I92E, I92G, I92L, E95K, E95N, E95Q, Y107N, D109N, T111S, S127A, S127E, S127F, or S127W.

In one embodiment, the IL-2 mutein described herein comprises: (i) an amino acid substitution at position L18, Q126, or S130; (ii) a replacement of the amino acid residues from positions N29 to L40 with an IL-15 hinge fragment-containing peptide; and (iii) an amino acid substitution at position E15, H16, D20, S87, N88, or 192.

In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 86; and (iii) an amino acid substitution of E15K, H16E, H16F, H16I, H16V, D20A, D20E, D20K, D20T, S87D, N88A, I92A, I92D, I92E, or I92G.

In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 86; and (iii) an amino acid substitution of E15K, H16E, H16F, H16I, H16V, D20A, D20E, D20K, D20T, S87D, N88A, I92A, I92D, I92E, or I92G.

In still another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 86; and (iii) an amino acid substitution of E15K, H16E, H16F, H16I, H16V, D20A, D20E, D20K, D20T, S87D, N88A, I92A, I92D, I92E, or I92G.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution at E15. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of E15K. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of E15K.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of H16. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of H16E, H16F, H16I, or H16V. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of H16E, H16F, H16I, or H16V.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of D20. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of D20A, D20E, D20K, or D20T. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of D20A, D20E, D20K, or D20T.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of S87. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of S87D. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of S87D.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of N88. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of N88A. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of N88A.

In one embodiment, the IL-2 mutein described herein comprises: (i) an amino acid substitution at position L18, Q126, or S130; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92. In another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A, I92D, I92E, or I92G. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A, I92D, I92E, or I92G. In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A, I92D, I92E, or I92G. In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A. In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92A. In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92D. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92D. In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92D. In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92E. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92E. In yet another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92E. In yet another embodiment, the IL-2 mutein described herein comprises: (i) an amino acid substitution of L18C, L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92G. In yet another embodiment, the IL-2 mutein described herein comprises: (i) an amino acid substitution of L18C, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86; and (iii) an amino acid substitution of I92G. In still another embodiment, the IL-2 mutein described herein comprises: (i) an amino acid substitution of L18S, Q126E, Q126K, Q126R, Q126S, Q126T, S130A, S130E, S130Q, or S130R; (ii) a replacement of the amino acid residues from positions N29 to L40 having the amino acid sequence of SEQ ID NO: 84 with an IL-1S hinge fragment-containing peptide having the amino acid sequence of SEQ ID NO: 85 or 86; and (iii) an amino acid substitution of I92G.

In one embodiment, each IL-2 domain in an anti-PD-1/IL-2 fusion protein provided herein independently comprises the amino acid sequence of any one of SEQ ID NOs: 46 to 83 and 87 to 156. In another embodiment, each IL-2 domain in an anti-PD-1/IL-2 fusion protein provided herein independently comprises the amino acid sequence of any one of SEQ ID NOs: 46 to 51. In yet another embodiment, each IL-2 domain in an anti-PD-1/IL-2 fusion protein provided herein is independently an IL-2 mutein comprising the amino acid sequence of any one of SEQ ID NOs: 52 to 83 and 87 to 156. In yet another embodiment, each IL-2 domain in an anti-PD-1/IL-2 fusion protein provided herein is independently an IL-2 mutein comprising the amino acid sequence of any one of SEQ ID NOs: 52 to 83 and 87 to 155. In yet another embodiment, each IL-2 domain in an anti-PD-1/IL-2 fusion protein provided herein is independently an IL-2 mutein comprising the amino acid sequence of any one of SEQ ID NOs: 52 to 83. In yet another embodiment, each IL-2 domain in an anti-PD-1/IL-2 fusion protein provided herein is independently an IL-2 mutein comprising the amino acid sequence of any one of SEQ ID NOs: 87 to 155. In still another embodiment, each IL-2 domain in an anti-PD-1/IL-2 fusion protein provided herein is independently an IL-2 mutein comprising the amino acid sequence of SEQ ID NO: 155.

In certain embodiments, the IL-2 mutein described herein has an attenuated binding affinity to an IL-2Rα as compared to a wild-type IL-2. In certain embodiments, the binding affinity of the IL-2 mutein described herein to an IL-2Rα is measured by its Kₐ, which is the inverse of its K_{d}.

In certain embodiments, the IL-2 mutein described herein has a K_{d} to the IL-2Rα of no less than about 2 times, no less than about 5 times, no less than about 10 times, no less than about 100 times, no less than about 200 times, no less than about 200 times, or no less than about 1,000 times higher than that of the wild-type IL-2 to the IL-2Rα. In one embodiment, the IL-2 mutein described herein has a K_{d} to the IL-2Rα of no less than about 2 times higher than that of the wild-type IL-2 to the IL-2Rα. In another embodiment, the IL-2 mutein described herein has a K_{d} to the IL-2Rα of no less than about 5 times higher than that of the wild-type IL-2 to the IL-2Rα. In yet another embodiment, the IL-2 mutein described herein has a K_{d} to the IL-2Rα of no less than about 10 times higher than that of the wild-type IL-2 to the IL-2Rα. In yet another embodiment, the IL-2 mutein described herein has a K_{d} to the IL-2Rα of no less than about 100 times higher than that of the wild-type IL-2 to the IL-2Rα. In yet another embodiment, the IL-2 mutein described herein has a K_{d} to the IL-2Rα of no less than about 200 times higher than that of the wild-type IL-2 to the IL-2Rα. In yet another embodiment, the IL-2 mutein described herein has a K_{d} to the IL-2Rα of no less than about 500 times higher than that of the wild-type IL-2 to the IL-2Rα. In still another embodiment, the IL-2 mutein described herein has a K_{d} to the IL-2Rα of no less than about 1,000 times higher than that of the wild-type IL-2 to the IL-2Rα.

In certain embodiments, the IL-2 mutein described herein has a K_{d} to an IL-2Rα of no less than about 20 nM, no less than about 50 nM, no less than about 100 nM, no less than about 1 μM, no less than about 10 M, no less than about 100 M, no less than about 200 μM, no less than about 500 μM, or no less than about 1 mM. In certain embodiments, the IL-2 mutein described herein has a K_{d} to the IL-2Rα of no less than about 20 nM. In certain embodiments, the IL-2 mutein described herein has a K_{d} to the IL-2Rα of no less than about 50 nM. In certain embodiments, the IL-2 mutein described herein has a K_{d} to the IL-2Rα of no less than about 100 nM. In certain embodiments, the IL-2 mutein described herein has a K_{d} to the IL-2Rα of no less than about 1 μM. In certain embodiments, the IL-2 mutein described herein has a K_{d} to the IL-2Rα of no less than about 10 μM. In certain embodiments, the IL-2 mutein described herein has a K_{d} to the IL-2Rα of no less than about 100 M. In certain embodiments, the IL-2 mutein described herein has a K_{d} to the IL-2Rα of no less than about 200 μM. In certain embodiments, the IL-2 mutein described herein has a K_{d} to the IL-2Rα of no less than about 500 μM. In certain embodiments, the IL-2 mutein described herein has a K_{d} to the IL-2Rα of no less than about 1 mM. In certain embodiments, the IL-2 mutein described herein has no measurable binding to the IL-2Rα. In certain embodiments, the IL-2 mutein described herein has no detectable binding to the IL-2Rα as measured with a surface plasmon resonance (SPR) method. In certain embodiments, the IL-2 mutein described herein has no detectable binding to the IL-2Rα as measured with bio-layer interferometry (BLI).

In certain embodiments, the IL-2 mutein described herein has a reduced binding affinity to an IL-2Rβ/γ complex as compared to a wild-type IL-2. In certain embodiments, the binding affinity of the IL-2 mutein described herein to an IL-2Rβ/γ complex is measured by its Kₐ, which is the inverse of its K_{d}.

In certain embodiments, the IL-2 mutein described herein has a K_{d} to the IL-2Rβ/γ complex of no less than about 5 times, no less than about 10 times, no less than about 20 times, no less than about 50 times, or no less than about 100 times higher than that of the wild-type IL-2 to the IL-2Rβ/γ complex. In one embodiment, the IL-2 mutein described herein has a K_{d} to the IL-2Rβ/γ complex of no less than about 5 times higher than that of the wild-type IL-2 to the IL-2Rβ/γ complex. In another embodiment, the IL-2 mutein described herein has a K_{d} to the IL-2Rβ/γ complex of no less than about 10 times higher than that of the wild-type IL-2 to the IL-2Rβ/γ complex. In yet another embodiment, the IL-2 mutein described herein has a K_{d} to the IL-2Rβ/γ complex of no less than about 20 times higher than that of the wild-type IL-2 to the IL-2Rβ/γ complex. In yet another embodiment, the IL-2 mutein described herein has a K_{d} to the IL-2Rβ/γ complex of no less than about 50 times higher than that of the wild-type IL-2 to the IL-2Rβ/γ complex. In still another embodiment, the IL-2 mutein described herein has a K_{d} to the IL-2Rβ/γ complex of no less than about 100 times higher than that of the wild-type IL-2 to the IL-2Rβ/γ complex.

In certain embodiments, the IL-2 mutein described herein has a K_{d} to an IL-2Rβ/γ complex of no less than about 5 nM, no less than about 10 nM, no less than about 20 nM, no less than about 50 nm, no less than about 100 nM, or no less than about 200 nM. In certain embodiments, the IL-2 mutein described herein has a K_{d} to the IL-2Rβ/γ complex of no less than about 5 nM. In certain embodiments, the IL-2 mutein described herein has a K_{d} to the IL-2Rβ/γ complex of no less than about 10 nM. In certain embodiments, the IL-2 mutein described herein has a K_{d} to the IL-2Rβ/γ complex of no less than about 20 nM. In certain embodiments, the IL-2 mutein described herein has a K_{d} to the IL-2Rβ/γ complex of no less than about 50 nM. In certain embodiments, the IL-2 mutein described herein has a K_{d} to the IL-2Rβ/γ complex of no less than about 100 nM. In certain embodiments, the IL-2 mutein described herein has a K_{d} to the IL-2Rβ/γ complex of no less than about 200 nM.

In one embodiment, the IL-2Rα is a human IL-2Rα. In another embodiment, the human IL-2Rα has the amino acid sequence of SEQ ID NO: 157.

In one embodiment, the IL-2Rβ is a human IL-2Rβ. In another embodiment, the human IL-2Rβ has the amino acid sequence of SEQ ID NO: 158.

In one embodiment, the IL-2Rγ is a human IL-2Rγ. In another embodiment, the human IL-2Rγ has the amino acid sequence of SEQ ID NO: 159.

In one embodiment, a K_{d} of an IL-2 to an IL-2Rα is determined with a surface plasmon resonance (SPR) method. In another embodiment, a K_{d} of an IL-2 to an IL-2Rα is determined with a BIACORE® assay. In yet another embodiment, a K_{d} of an IL-2 to an IL-2Rα is determined with bio-layer interferometry (BLI). In still another embodiment, a K_{d} of an IL-2 to an IL-2Rα is determined with an OCTET® assay.

In one embodiment, a K_{d} of an IL-2 to an IL-2Rβ is determined with an SPR method. In another embodiment, a K_{d} of an IL-2 to an IL-2Rβ is determined with a BIACORE® assay. In yet another embodiment, a K_{d} of an IL-2 to an IL-2Rβ is determined with BLI. In still another embodiment, a K_{d} of an IL-2 to an IL-2Rβ is determined with an OCTET® assay.

In one embodiment, a K_{d} of an IL-2 to an IL-2Rβ/γ complex is determined with an SPR method. In another embodiment, a K_{d} of an IL-2 to an IL-2Rβ/γ complex is determined with a BIACORE® assay. In yet another embodiment, a K_{d} of an IL-2 to an IL-2Rβ/γ complex is determined with BLI. In still another embodiment, a K_{d} of an IL-2 to an IL-2Rβ/γ complex is determined with an OCTET® assay.

In one embodiment, a K_{d} of an IL-2 to an IL-2Rα/β/γ complex is determined with an SPR method. In another embodiment, a K_{d} of an IL-2 to an IL-2Rα/β/γ complex is determined with a BIACORE® assay. In yet another embodiment, a K_{d} of an IL-2 to an IL-2Rα/β/γ complex is determined with BLI. In still another embodiment, a K_{d} of an IL-2 to an IL-2Rα/β/γ complex is determined with an OCTET® assay.

In certain embodiments, the IL-2 mutein described herein comprises an amino acid sequence that is no less than about 80%, no less than about 85%, no less than about 90%, no less than about 91%, no less than about 92%, no less than about 93%, no less than about 94%, no less than about 95%, no less than about 96%, no less than about 97%, no less than about 98%, or no less than about 99% identical to the amino acid sequence of SEQ ID NO: 46, 47, 48, 49, 50, or 51. In certain embodiments, the IL-2 mutein described herein comprises an amino acid sequence that is no less than about 80% identical to the amino acid sequence of SEQ ID NO: 46, 47, 48, 49, 50, or 51. In certain embodiments, the IL-2 mutein described herein comprises an amino acid sequence that is no less than about 85% identical to the amino acid sequence of SEQ ID NO: 46, 47, 48, 49, 50, or 51. In certain embodiments, the IL-2 mutein described herein comprises an amino acid sequence that is no less than about 90% identical to the amino acid sequence of SEQ ID NO: 46, 47, 48, 49, 50, or 51. In certain embodiments, the IL-2 mutein described herein comprises an amino acid sequence that is no less than about 91% identical to the amino acid sequence of SEQ ID NO: 46, 47, 48, 49, 50, or 51. In certain embodiments, the IL-2 mutein described herein comprises an amino acid sequence that is no less than about 92% identical to the amino acid sequence of SEQ ID NO: 46, 47, 48, 49, 50, or 51. In certain embodiments, the IL-2 mutein described herein comprises an amino acid sequence that is no less than about 93% identical to the amino acid sequence of SEQ ID NO: 46, 47, 48, 49, 50, or 51. In certain embodiments, the IL-2 mutein described herein comprises an amino acid sequence that is no less than about 94% identical to the amino acid sequence of SEQ ID NO: 46, 47, 48, 49, 50, or 51. In certain embodiments, the IL-2 mutein described herein comprises an amino acid sequence that is no less than about 95% identical to the amino acid sequence of SEQ ID NO: 46, 47, 48, 49, 50, or 51 In certain embodiments, the IL-2 mutein described herein comprises an amino acid sequence that is no less than about 96% identical to the amino acid sequence of SEQ ID NO: 46, 47, 48, 49, 50, or 51. In certain embodiments, the IL-2 mutein described herein comprises an amino acid sequence that is no less than about 97% identical to the amino acid sequence of SEQ ID NO: 46, 47, 48, 49, 50, or 51. In certain embodiments, the IL-2 mutein described herein comprises an amino acid sequence that is no less than about 98% identical to the amino acid sequence of SEQ ID NO: 46, 47, 48, 49, 50, or 51. In certain embodiments, the IL-2 mutein described herein comprises an amino acid sequence that is no less than about 99% identical to the amino acid sequence of SEQ ID NO: 46, 47, 48, 49, 50, or 51.

In certain embodiments, the IL-2 domain in an anti-PD-1/IL-2 fusion protein provided herein is an IL-2 mutein disclosed in CN 111018961 A, US 2018/0326010 A1, WO 2020/005819 A1, WO 2021/102063 A1, WO 2021/222150 A2, and WO 2022/212614 A1, the disclosure of each of which is incorporated herein by reference in its entirety.

In certain embodiments, the IL-2 mutein described herein further includes one or more additional substitutions, deletions, and/or insertions; and/or one or more additional post-translational modifications.

In one embodiment, each peptide linker in a fusion protein provided herein independently comprises the amino acid sequence of GSG or one of SEQ ID NOs: 6 to 45. In another embodiment, each peptide linker in a fusion protein provided herein independently comprises the amino acid sequence of GSG or SEQ ID NO: 6, 7, or 8. In yet another embodiment, each peptide linker in a fusion protein provided herein independently comprises the amino acid sequence of SEQ ID NO: 9, 10, 11, or 12. In yet another embodiment, each peptide linker in a fusion protein provided herein independently comprises the amino acid sequence of SEQ ID NO: 13, 14, 15, or 16. In yet another embodiment, each peptide linker in a fusion protein provided herein independently comprises the amino acid sequence of SEQ ID NO: 17, 18, 19, or 20. In yet another embodiment, each peptide linker in a fusion protein provided herein independently comprises the amino acid sequence of SEQ ID NO: 21, 22, 23, or 24. In yet another embodiment, each peptide linker in a fusion protein provided herein independently comprises the amino acid sequence of SEQ ID NO: 25, 26, 27, or 28. In yet another embodiment, each peptide linker in a fusion protein provided herein independently comprises the amino acid sequence of SEQ ID NO: 29, 30, 31, or 32. In yet another embodiment, each peptide linker in a fusion protein provided herein independently comprises the amino acid sequence of SEQ ID NO: 33, 34, 35, or 36. In yet another embodiment, each peptide linker in a fusion protein provided herein independently comprises the amino acid sequence of SEQ ID NO: 37. In yet another embodiment, each peptide linker in a fusion protein provided herein independently comprises the amino acid sequence of SEQ ID NO: 38 or 39. In yet another embodiment, each peptide linker in a fusion protein provided herein independently comprises the amino acid sequence of SEQ ID NO. 40 or 41. In yet another embodiment, each peptide linker in a fusion protein provided herein independently comprises the amino acid sequence of SEQ ID NO: 42 or 43. In still another embodiment, each peptide linker in a fusion protein provided herein independently comprises the amino acid sequence of SEQ ID NO: 44 or 45.

In one embodiment, provided herein is an anti-PD-1/IL-2 fusion protein comprising the amino acid sequence of SEQ ID NO: 160 and/or 161. In another embodiment, provided herein is an anti-PD-1/IL-2 fusion protein comprising the amino acid sequence of SEQ ID NO: 160. In yet another embodiment, provided herein is an anti-PD-1/IL-2 fusion protein comprising the amino acid sequence of SEQ ID NO: 161. In still another embodiment, provided herein is an anti-PD-1/IL-2 fusion protein comprising the amino acid sequences of SEQ ID NOs: 160 and 161.

### Pharmaceutical Compositions

In one embodiment, provided herein is a pharmaceutical composition comprising an anti-PD-1/IL-2 fusion protein provided herein and a pharmaceutically acceptable excipient.

In one embodiment, the pharmaceutical composition is formulated as single dosage form.

In one embodiment, the pharmaceutical composition provided herein is a solid formulation. In another embodiment, the pharmaceutical composition provided herein is a lyophilized solid formulation. In yet another embodiment, the pharmaceutical composition provided herein is a solution. In still another embodiment, the pharmaceutical composition provided herein is an aqueous solution.

In one embodiment, the pharmaceutical composition provided herein is formulated in a dosage form for parenteral administration. In another embodiment, the pharmaceutical composition provided herein is formulated in a dosage form for intravenous administration. In yet another embodiment, the pharmaceutical composition provided herein is formulated in a dosage form for intramuscular administration. In yet another embodiment, the pharmaceutical composition provided herein is formulated in a dosage form for subcutaneous administration. In still another embodiment, the pharmaceutical composition provided herein is formulated in a dosage form for intratumoral administration.

### Methods of Use

In one embodiment, provided herein is a method for treating, preventing, or ameliorating a proliferative disease in a subject, comprising administering to the subject in need thereof a therapeutically effective amount of an anti-PD-1/IL-2 fusion protein provided herein.

In one embodiment, the proliferative disease is cancer. In another embodiment, the proliferative disease is colon cancer or colorectal cancer.

In certain embodiments, the cancer is refractory and/or relapsed. In certain embodiments, the cancer is refractory. In certain embodiments, the cancer is relapsed. In certain embodiments, the cancer is metastatic. In certain embodiments, the cancer is resectable. In certain embodiments, the cancer is unresectable. In certain embodiments, the cancer is metastatic.

In certain embodiments, the cancer is drug-resistant. In certain embodiments, the cancer is multidrug-resistant. In certain embodiments, the cancer is resistant to a chemotherapy. In certain embodiments, the cancer is resistant to an immunotherapy. In certain embodiments, the cancer is resistant to a standard therapy for the cancer.

In certain embodiments, the subject is a mammal. In certain embodiments, the subject is a human.

In another embodiment, provided herein is a method of inhibiting the growth of a cell, comprising contacting the cell with an effective amount of an anti-PD-1/IL-2 fusion protein provided herein. In certain embodiments, the cell is a cancerous cell. In certain embodiments, the cell is a human cancerous cell. In certain embodiments, the cell is a metastatic cancerous cell.

In certain embodiments, the therapeutically effective amount of an anti-PD-1/IL-2 fusion protein provided herein is ranging from about 0.001 mg per kg subject body weight every month (mg/kg per month) to 100 mg per kg subject body weight per day (mg/kg per day), from about 0.01 mg/kg per month to about 75 mg/kg per day, from about 0.1 mg/kg per month to about 50 mg/kg per day, from about 0.5 mg/kg per month to about 25 mg/kg per day, or from about 1 mg/kg per month to about 20 mg/kg per day, which can be administered in single or multiple doses. Within this range, the dosage can be ranging from about 0.005 mg/kg per month to about 0.05 mg/kg per day, from about 0.05 mg/kg per month to about 0.5 mg/kg per day, from about 0.5 mg/kg per month to about 5.0 mg/kg per day, from about 1 mg/kg per month to about 15 mg/kg per day, from about 1 mg/kg per month to about 20 mg/kg per day, or from about 1 mg/kg per month to about 50 mg/kg per day.

The disclosure will be further understood by the following non-limiting examples.

### EXAMPLES

### Example 1

### Cloning, Expression, and Purification of Fusion Proteins

The amino acid sequence of human IL-2 (hIL-2) was obtained from UNIPROT (hIL-2: P60568, 21-153 aa). A mutant hIL-2 was generated by introducing a mutation to attenuating CD25 and CD122/CD132 bindings. The deoxyoligonucleotide (DNA) sequences encoding the mutant hIL-2, a human anti-PD-1 single domain antibody, and a peptide chain of a Fc domain were codon optimized for CHO cell expression.

The DNA sequences encoding (i) the hIL-2 mutein, (ii) the human anti-PD-1 single domain antibody, (iii) a peptide chain of the Fc domain, and (iv) a peptide linker were seamlessly assembled together by homology assembly cloning with commercially available kits. The oligonucleotides of each fusion protein were inserted into a UCOE® expression vector CET1019-AS-Puro for CHO cell expression.

Each fusion protein produced in the CHO cells was purified by a two-step purification process: protein A affinity chromatography using protein A (e.g., AMSPHERE™ A3 or MABSELECT™ SURE™) resin, and ion exchange chromatography (e.g., CAPTO™ Q IMPRES or CAPTO™ S IMPACT) or hydrophobic interaction chromatography (e.g., Phenyl HP).

### Example 2

### Binding Studies of IL-2 Muteins to CD122 and CD122/CD132

OCTET® RED96 was used to characterize the interactions of wild-type hIL-2 and hIL-2 muteins with CD25, CD122, or CD122/CD132 heterodimer. CD122 and AVITAG CD122/CD132 heterodimer were purchased from ACROBIOSYSTEMS, Inc. The protein samples were diluted in the OCTET® kinetic buffer. Briefly, hIL-2 receptors were loaded onto a biosensor. The biosensor was then dipped into a solution containing a wild-type hIL-2 and IL-2 muteins at 10 to 2,000 nM. The association and dissociation sensorgram was fitted by global or local fitting using the OCTET® Data Analysis HT software. The results are summarized in Table 1. All the hIL-2 muteins tested were found to have abolished or significantly reduced CD25 binding.

TABLE 1
Interactions of Wild-type hIL-2 and hIL-2
Muteins with CD122 or CD122/CD132

| | SEQ | K_{d} (nM) | |
|---|---|---|---|
| Fusion Protein | ID NO: | CD122 | CD122/CD132 |
| WT IL-2 | 46 | | 1.9 |
| hIL-2 Mutein | 133 | | 1 |
| (T3A, R38E, F42A, K76E, C125S) | | | |
| IL-2-IL-15 Chimera 2 | 88 | 104 | 11 |
| (T3A, C125S) | | | |
| IL-2-IL-15 Chimera 29 | 115 | | 3.5 |
| (I92A, C125S) | | | |
| IL-2-IL-15 Chimera 42 | 128 | | 53 |
| (E15K, N88A, C125S) | | | |
| IL-2-IL-15 Chimera 44 | 130 | | 12 |
| (T3A, L18C) | | | |
| IL-2-IL-15 Chimera 46 | 132 | | 219 |
| (T3A, C125S, Q126E) | | | |
| IL-2-IL-15 Chimera 54 | 140 | 650 | 30 |
| (T3A, C125S, Q126T) | | | |
| IL-2-IL-15 Chimera 56 | 142 | 808 | 12 |
| (T3A, C125S, S130R) | | | |
| IL-2-IL-15 Chimera 58 | 144 | 188 | 229 |
| (T3A, L18C, Q126E) | | | |
| IL-2-IL-15 Chimera 64 | 150 | | 382 |
| (T3A, L18C, Q126S) | | | |
| IL-2-IL-15 Chimera 65 | 151 | | 242 |
| (T3A, I92A, C125S, Q126E) | | | |
| IL-2-IL-15 Chimera 68 | 154 | 466 | 32 |
| (T3A, C125S, Q126T, S130R) | | | |
| IL-2-IL-15 Chimera 69 | 155 | | 322 |
| (T3A, L18S, C125S, Q126E) | | | |
| hIL-2 Mutein | 156 | 550 | 6.8 |
| (T3A, F42A, Y45A, L72G, C125S) | | | |

### Example 3

### Antitumor Activity of an Anti-PD-1/IL-2 Fusion Protein in a Xenograft Mouse Model

MC38 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 4

### Antitumor Activity of an Anti-PD-1/IL-2 Fusion Protein in a Xenograft Model Using PD-1 Knock-in Mice

MC38 cells or B16F10 cells are cultured and maintained in DMEM media supplemented with 10% fetal bovine serum, GLUTAMAX™, non-essential amino acids (NEAA), sodium pyruvate, and penicillin/streptomycin. The cells are trypsinized, washed with the media, and counted. The cells are diluted with PBS and 5×10⁵ cells in PBS (50 μL) are injected subcutaneously into anesthetized C57BL/6 mice with human PD-1 knock-in using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 5

### Antitumor Activity of an Anti-PD-1/IL-2 Fusion Protein in a Xenograft Mouse Model

CT26 mouse cells are cultured and maintained in RPMI media supplemented with 10% fetal bovine serum, GLUTAMAX™, and penicillin/streptomycin. The cells are trypsinized, washed with media, and counted. The cells are diluted with PBS and 1×10⁶ cells in PBS (100 μL) are injected subcutaneously into anesthetized BALB/c mice using an 18-gauge needle. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 6

### Antitumor Activity of an Anti-PD-1/IL-2 Fusion Protein in a Xenograft Mouse Model

HT-29 cells are cultured and maintained in McCoys 5a media supplemented with 10% fetal bovine serum and penicillin/streptomycin. The cells are trypsinized, washed with media, counted, and washed with PBS. The cell suspension (1×10⁶ cells in PBS (100 μL)) is injected subcutaneously into anesthetized NCG mice using a 27-gauge needle. After 6 days, human PBMCs (1×10⁷ cells in PBS (100 μL)) are injected into the tail vein of each mouse. A stock solution of an anti-PD-1/IL-2 fusion protein is diluted in PBS on the day of dosing and the mice are dosed intraperitoneally with PBS (control) or the anti-PD-1/IL-2 fusion protein in PBS twice a week for two weeks. Tumor sizes (length (L) and width (W)) are measured twice per week using a digital caliper, and the tumor volume is calculated (L×W×W)/2.

### Example 7

### Effect of an Anti-PD-1/IL-2 Fusion Proteins on STAT5 Signaling

Anti-PD-1/IL-2 fusion proteins are evaluated for their effect on pSTAT5 signaling via its IL-2 domain in CD3 T-cells. Briefly, CD3 T-cells (100,000) are treated with an anti-PD-1/IL-2 fusion protein for 30 min at 37° C. and 5% CO₂ in Hanks balanced salt solution containing 10 mM HEPES. Phospho-STAT5 is measured using a phosphor-STAT5 (Tyr694) HTRF assay. The signal ratio at 665 nm/620 nm is multiplied by 1,000 and the data is analyzed with global fitting to determine EC₅₀ values.

### Example 8

### Binding Studies of Anti-PD-1/IL-2 Fusion Proteins to Human PD-1 and IL-2 Receptors

Anti-PD-1/IL-2 fusion protein A1 was made by co-expression of SEQ ID NOs: 160 and 161 in CHO cells. OCTET® RED96 was used to study the interaction between anti-PD-1/IL-2 fusion protein A1 and human PD-1 or IL-2 receptors. For the binding study of the human PD-1, anti-human-Fc (AHC) biosensors were used to capture anti-PD-1/IL-2 fusion protein A1. The coated biosensors were associated with serially diluted His-tagged human PD-1 protein at predetermined concentrations. For the binding study of human IL-2 receptors, anti-penta-HIS (HIS1K) biosensors were used to capture human IL-2Rα or human IL-2Rβγ (His) proteins. The coated biosensors were associated with serially diluted anti-PD-1/IL-2 fusion protein A1 at predetermined concentrations.

The His-tagged IL-2Rα (CD25) protein, His-tagged & Twin Strep-tagged human IL-2Rβ & IL-2Rγ heterodimer protein, His-tagged human PD-1, and recombinant human IL-2 (rhIL-2) protein were purchased form ACROBIOSYSTEMS, Inc. The association and dissociation sensorgrams were fitted by global or local fitting using the OCTET® Data Analysis HT software. The results are summarized in Table 2.

TABLE 2
Interactions of Fusion Protein A1 and rhIL-2
with Human IL-2 Receptors and Human PD-1

| Receptor | Compound | K_{D} (nM) |
|---|---|---|
| Human IL-2Rα | Fusion Protein A1 | Not Detected |
| | rhIL-2 | 26 |
| Human IL-2Rβγ | Fusion Protein A1 | 12 |
| | rhIL-2 | 0.43 |
| Human PD-1 | Fusion Protein A1 | 5 |
| | rhIL-2 | Not Detected |

Sequences described herein are provided in the sequence table below.

SEQUENCE TABLE

| SEQ ID NO: | Description | Amino Acid Sequence |
|---|---|---|
| 1 | Anti-hPD-1-V_{H}H CDR1 | GRTYA |
| | (IMGT) | |
| 2 | Anti-hPD-1-V_{H}H CDR2 | IRWSSTFT |
| | (IMGT) | |
| 3 | Anti-hPD-1-V_{H}H CDR3 | AAQTSQRGTLYD |
| | (IMGT) | |
| 4 | Anti-hPD-1-V_{H}H-A | QVQLLESGGGLVKPGGSLRLSCAASGRTYAMGWF |
| | | RRAPGKESEFVAEIRWSSTFTDYADSVKGRFTISRD |
| | | NAENSAYLQMNSLRAEDTAVYHCAAQTSQRGTLY |
| | | DSYGQGTQVTVSS |
| 5 | Anti-hPD-1-V_{H}H-B | QVQLVESGGGVVQPGGSLRLSCAASGRTYAMGWF |
| | | RQAPGKESEFVAEIRWSGSFTYYADSVKGRFTISRD |
| | | NSENTAYLQMNSLRAEDTAVYRCAAQTSQRGTLY |
| | | DHYGQGTLVTVSS |
| | GSG Linker | GSG |
| 6 | (GSG)₂ Linker | GSGGSG |
| 7 | (GSG)₃ Linker | GSGGSGGSG |
| 8 | (GSG)₄ Linker | GSGGSGGSGGSG |
| 9 | G3S Linker | GGGS |
| 10 | (G3S)₂ Linker | GGGSGGGS |
| 11 | (G3S)₃ Linker | GGGSGGGSGGGS |
| 12 | (G3S)₄ Linker | GGGSGGGSGGGSGGGS |
| 13 | G4S Linker | GGGGS |
| 14 | (G4S)₂ Linker | GGGGSGGGGS |
| 15 | (G4S)₃ Linker | GGGGSGGGGSGGGGS |
| 16 | (G4S)₄ Linker | GGGGSGGGGSGGGGSGGGGS |
| 17 | SGSG Linker | SGSG |
| 18 | S(GSG)₂ Linker | SGSGGSG |
| 19 | S(GSG)₃ Linker | SGSGGSGGSG |
| 20 | S(GSG)₄ Linker | SGSGGSGGSGGSG |
| 21 | SG3S Linker | SGGGS |
| 22 | S(G3S)₂ Linker | SGGGSGGGS |
| 23 | S(G3S)₃ Linker | SGGGSGGGSGGGS |
| 24 | S(G3S)₄ Linker | SGGGSGGGSGGGSGGGS |
| 25 | SG4S Linker | SGGGGS |
| 26 | S(G4S)₂ Linker | SGGGGSGGGGS |
| 27 | S(G4S)₃ Linker | SGGGGSGGGGSGGGGS |
| 28 | S(G4S)₄ Linker | SGGGGSGGGGSGGGGSGGGGS |
| 29 | EAAAK Linker | EAAAK |
| 30 | (EAAAK)₂ Linker | EAAAKEAAAK |
| 31 | (EAAAK)₃ Linker | EAAAKEAAAKEAAAK |
| 32 | (EAAAK)₄ Linker | EAAAKEAAAKEAAAKEAAAK |
| 33 | PAPAP Linker | PAPAP |
| 34 | (PAPAP)₂ Linker | PAPAPPAPAP |
| 35 | (PAPAP)₃ Linker | PAPAPPAPAPPAPAP |
| 36 | (PAPAP)₄ Linker | PAPAPPAPAPPAPAPPAPAP |
| 37 | VLVH Linker | IKRTVAAP |
| 38 | RAKPS Linker | RAKPS |
| 39 | (RAKPS)₂ Linker | RAKPSRAKPS |
| 40 | ASTKG Linker | ASTKG |
| 41 | (ASTKG)₂ Linker | ASTKGASTKG |
| 42 | AKTHT Linker | AKTHT |
| 43 | (AKTHT)₂ Linker | AKTHTAKTHT |
| 44 | ASTKG(G4S)₂ Linker | ASTKGGGGGSGGGGS |
| 45 | RAKPS(G4S)₂ Linker | RAKPSGGGGSGGGGS |
| 46 | hIL-2 | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFCQSIISTLT |
| 47 | hIL-2 (T3A) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFCQSIISTLT |
| 48 | hIL-2 (C125S) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 49 | hIL-2 (C125A) | APTSSSTKKTQLQLEHLLLDLQMILNGINNYKNPKL |
| | | TRMLTFKFYMPKKATELKHLQCLEEELKPLEEVLN |
| | | LAQSKNFHLRPRDLISNINVIVLELKGSETTFMCEY |
| | | ADETATIVEFLNRWITFAQSIISTLT |
| 50 | hIL-2 (T3A, C125S) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 51 | hIL-2 (T3A, C125A) | APASSSTKKTQLQLEHLLLDLQMILNGINNYKNPK |
| | | LTRMLTFKFYMPKKATELKHLQCLEEELKPLEEVL |
| | | NLAQSKNFHLRPRDLISNINVIVLELKGSETTFMCE |
| | | YADETATIVEFLNRWITFAQSIISTLT |
| 52 | hIL-2 Mutein | APTSSSTKKTQLQLEHLLLDLQMILNGINNYKNPKL |
| | (R38N, L40T, C125S) | TNMTTFKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 53 | hIL-2 Mutein | APASSSTKKTQLQLEHLLLDLQMILNGINNYKNPK |
| | (T3A, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 54 | hIL-2 Mutein | APTSSSTKKTQLQLKHLLLDLQMILNGINNYKNPK |
| | (E15K, R38N, L40T, C125S) | LTNMTTFKFYMPKKATELKHLQCLEEELKPLEEVL |
| | | NLAQSKNFHLRPRDLISNINVIVLELKGSETTFMCE |
| | | YADETATIVEFLNRWITFSQSIISTLT |
| 55 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | C125S) | NLAQSKNFHLRPRDLISNINVIVLELKGSETTFMCE |
| | | YADETATIVEFLNRWITFSQSIISTLT |
| 56 | hIL-2 Mutein | APTSSSTKKTQLQLKHLLLDLQMILNGINNYENPKL |
| | (E15K, K32E, R38N, L40T, | TNMTTFKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 57 | hIL-2 Mutein | APASSSTKKTQLQLKHLLLDLQMILNGINNYENPK |
| | (T3A, E15K, K32E, R38N, | LTNMTTFKFYMPKKATELKHLQCLEEELKPLEEVL |
| | L40T, C125S) | NLAQSKNFHLRPRDLISNINVIVLELKGSETTFMCE |
| | | YADETATIVEFLNRWITFSQSIISTLT |
| 58 | hIL-2 Mutein | APTSSSTKKTQLQLKHLLLDLQMILNGINNYKNPK |
| | (E15K, R38N, L40T, K76E, | LTNMTTFKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 59 | hIL-2 Mutein | APASSSTKKTQLQLKHLLLDLQMILNGINNYKNPK |
| | (T3A, E15K, R38N, L40T, | LTNMTTFKFYMPKKATELKHLQCLEEELKPLEEVL |
| | K76E, C125S) | NLAQSENFHLRPRDLISNINVIVLELKGSETTFMCE |
| | | YADETATIVEFLNRWITFSQSIISTLT |
| 60 | hIL-2 Mutein | APTSSSTKKTQLQLEHLLLDLQMILNGINNYENPKL |
| | (K32E, R38E, F42A, C125S) | TEMLTAKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 61 | hIL-2 Mutein | APASSSTKKTQLQLEHLLLDLQMILNGINNYENPKL |
| | (T3A, K32E, R38E, F42A, | TEMLTAKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 62 | hIL-2 Mutcin | APTSSSTKKTQLQLEHLLLDLQMILNGINNYENPKL |
| | (K32E, R38E, F42K, C125S) | TEMLTKKFYMPKKATELKHLQCLEEELKPLEEVLN |
| | | LAQSKNFHLRPRDLISNINVIVLELKGSETTFMCEY |
| | | ADETATIVEFLNRWITFSQSIISTLT |
| 63 | hIL-2 Mutein | APASSSTKKTQLQLEHLLLDLQMILNGINNYENPKL |
| | (T3A, K32E, R38E, F42K, | TEMLTKKFYMPKKATELKHLQCLEEELKPLEEVLN |
| | C125S) | LAQSKNFHLRPRDLISNINVIVLELKGSETTFMCEY |
| | | ADETATIVEFLNRWITFSQSIISTLT |
| 64 | hIL-2 Mutein | APTSSSTKKTQLQLKHLLLDLQMILNGINNYENPKL |
| | (E15K, K32E, R38E, F42K, | TEMLTKKFYMPKKATELKHLQCLEEELKPLEEVLN |
| | | LAQSKNFHLRPRDLISNINVIVLELKGSETTFMCEY |
| | C125S) | ADETATIVEFLNRWITFSQSIISTLT |
| 65 | hIL-2 Mutein | APASSSTKKTQLQLKHLLLDLQMILNGINNYENPK |
| | (T3A, E15K, K32E, R38E, | LTEMLTKKFYMPKKATELKHLQCLEEELKPLEEVL |
| | F42K, C125S) | NLAQSKNFHLRPRDLISNINVIVLELKGSETTFMCE |
| | | YADETATIVEFLNRWITFSQSIISTLT |
| 66 | hIL-2 Mutein | APTSSSTKKTQLQLEHLLLDLQMILNGINNYKNPKL |
| | (R38E, F42A, K76E, C125S) | TEMLTAKFYMPKKATELKHLQCLEEELKPLEEVLN |
| | | LAQSENFHLRPRDLISNINVIVLELKGSETTFMCEY |
| | | ADETATIVEFLNRWITFSQSIISTLT |
| 67 | hIL-2 Mutein | APASSSTKKTQLQLEHLLLDLQMILNGINNYKNPK |
| | (T3A, R38E, F42A, K76E, | LTEMLTAKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 68 | hIL-2 Mutein | APTSSSTKKTQLQLEHLLLDLQMILNGINNYKNPKL |
| | (R38E, F42K, K76E, C125S) | TEMLTKKFYMPKKATELKHLQCLEEELKPLEEVLN |
| | | LAQSENFHLRPRDLISNINVIVLELKGSETTFMCEY |
| | | ADETATIVEFLNRWITFSQSIISTLT |
| 69 | hIL-2 Mutein | APASSSTKKTQLQLEHLLLDLQMILNGINNYKNPK |
| | (T3A, R38E, F42K, K76E, | LTEMLTKKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 70 | hIL-2 Mutein | APTSSSTKKTQLQLKHLLLDLQMILNGINNYKNPK |
| | (E15K, R38E, F42K, K76E, | LTEMLTKKFYMPKKATELKHLQCLEEELKPLEEVL |
| | C125S) | NLAQSENFHLRPRDLISNINVIVLELKGSETTFMCE |
| | | YADETATIVEFLNRWITFSQSIISTLT |
| 71 | hIL-2 Mutein | APASSSTKKTQLQLKHLLLDLQMILNGINNYKNPK |
| | (T3A, E15K, R38E, F42K, | LTEMLTKKFYMPKKATELKHLQCLEEELKPLEEVL |
| | K76E, C125S) | NLAQSENFHLRPRDLISNINVIVLELKGSETTFMCE |
| | | YADETATIVEFLNRWITFSQSIISTLT |
| 72 | hIL-2 Mutein | APTSSSTKKTQLQLEHLLLDLQMILNGINNYKNPKL |
| | (F42C, V69C, C125S) | TRMLTCKFYMPKKATELKHLQCLEEELKPLEECLN |
| | | LAQSKNFHLRPRDLISNINVIVLELKGSETTFMCEY |
| | | ADETATIVEFLNRWITFSQSIISTLT |
| 73 | hIL-2 Mutcin | APASSSTKKTQLQLEHLLLDLQMILNGINNYKNPK |
| | (T3A, F42C, V69C, C125S) | LTRMLTCKFYMPKKATELKHLQCLEEELKPLEECL |
| | | NLAQSKNFHLRPRDLISNINVIVLELKGSETTFMCE |
| | | YADETATIVEFLNRWITFSQSIISTLT |
| 74 | hIL-2 Mutein | APTSSSTKKTQLQLEHLLLDLQMILNGINNYENPKL |
| | (K32E, F42C, V69C, C125S) | TRMLTCKFYMPKKATELKHLQCLEEELKPLEECLN |
| | | LAQSKNFHLRPRDLISNINVIVLELKGSETTFMCEY |
| | | ADETATIVEFLNRWITFSQSIISTLT |
| 75 | hIL-2 Mutein | APASSSTKKTQLQLEHLLLDLQMILNGINNYENPKL |
| | (T3A, K32E, F42C, V69C, | TRMLTCKFYMPKKATELKHLQCLEEELKPLEECLN |
| | | LAQSKNFHLRPRDLISNINVIVLELKGSETTFMCEY |
| | C125S) | ADETATIVEFLNRWITFSQSIISTLT |
| 76 | hIL-2 Mutein | APTSSSTKKTQLQLEHLLLDLQMILNGINNYKNPKL |
| | (F42C, V69C, K76E, C125S) | TRMLTCKFYMPKKATELKHLQCLEEELKPLEECLN |
| | | LAQSENFHLRPRDLISNINVIVLELKGSETTFMCEY |
| | | ADETATIVEFLNRWITFSQSIISTLT |
| 77 | hIL-2 Mutein | APASSSTKKTQLQLEHLLLDLQMILNGINNYKNPK |
| | (T3A, F42C, V69C, K76E, | LTRMLTCKFYMPKKATELKHLQCLEEELKPLEECL |
| | C125S) | NLAQSENFHLRPRDLISNINVIVLELKGSETTFMCE |
| | | YADETATIVEFLNRWITFSQSIISTLT |
| 78 | hIL-2 Mutein | APTSSSTKKTQLQLKHLLLDLQMILNGINNYKNPK |
| | (E15K, F42C, V69C, C125S) | LTRMLTCKFYMPKKATELKHLQCLEEELKPLEECL |
| | | NLAQSKNFHLRPRDLISNINVIVLELKGSETTFMCE |
| | | YADETATIVEFLNRWITFSQSIISTLT |
| 79 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | C125S) | NLAQSKNFHLRPRDLISNINVIVLELKGSETTFMCE |
| | | YADETATIVEFLNRWITFSQSIISTLT |
| 80 | hIL-2 Mutein | APTSSSTKKTQLQLKHLLLDLQMILNGINNYENPKL |
| | (E15K, K32E, F42C, V69C, | TRMLTCKFYMPKKATELKHLQCLEEELKPLEECLN |
| | C125S) | LAQSKNFHLRPRDLISNINVIVLELKGSETTFMCEY |
| | | ADETATIVEFLNRWITFSQSIISTLT |
| 81 | hIL-2 Mutein | APASSSTKKTQLQLKHLLLDLQMILNGINNYENPK |
| | (T3A, E15K, K32E, F42C, | LTRMLTCKFYMPKKATELKHLQCLEEELKPLEECL |
| | V69C, C125S) | NLAQSKNFHLRPRDLISNINVIVLELKGSETTFMCE |
| | | YADETATIVEFLNRWITFSQSIISTLT |
| 82 | hIL-2 Mutein | APTSSSTKKTQLQLKHLLLDLQMILNGINNYKNPK |
| | (E15K, F42C, V69C, K76E, | LTRMLTCKFYMPKKATELKHLQCLEEELKPLEECL |
| | C125S) | NLAQSENFHLRPRDLISNINVIVLELKGSETTFMCE |
| | | YADETATIVEFLNRWITFSQSIISTLT |
| 83 | hIL-2 Mutein | APASSSTKKTQLQLKHLLLDLQMILNGINNYKNPK |
| | (T3A, E15K, F42C, V69C, | LTRMLTCKFYMPKKATELKHLQCLEEELKPLEECL |
| | K76E, C125S) | NLAQSENFHLRPRDLISNINVIVLELKGSETTFMCE |
| | | YADETATIVEFLNRWITFSQSIISTLT |
| 84 | hIL-2 Fragment | NNYKNPKLTRML |
| | (N29 to L40) | |
| 85 | IL-15 Hinge Fragment- | QSMEIDAT |
| | containing Peptide 1 | |
| 86 | IL-15 Hinge Fragment- | QSMHIDAT |
| | containing peptide 2 | |
| 87 | IL-2-IL-15 Chimera 1 | APTSSSTKKTQLQLEHLLLDLQMILNGIQSMEIDAT |
| | (C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 88 | IL-2-IL-15 Chimera 2 | APASSSTKKTQLQLEHLLLDLQMILNGIQSMEIDAT |
| | (T3A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 89 | IL-2-IL-15 Chimera 3 | APTSSSTKKTQLQLEHLLLDLQMILNGIQSMHIDAT |
| | (E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 90 | IL-2-IL-15 Chimera 4 | APASSSTKKTQLQLEHLLLDLQMILNGIQSMHIDAT |
| | (T3A, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 91 | IL-2-IL-15 Chimera 5 | APTSSSTKKTQLQLKHLLLDLQMILNGIQSMEIDAT |
| | (E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 92 | IL-2-IL-15 Chimera 6 | APASSSTKKTQLQLKHLLLDLQMILNGIQSMEIDAT |
| | (T3A, E15K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 93 | IL-2-IL-15 Chimera 7 | APTSSSTKKTQLQLKHLLLDLQMILNGIQSMHIDAT |
| | (E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 94 | IL-2-IL-15 Chimera 8 | APASSSTKKTQLQLKHLLLDLQMILNGIQSMHIDAT |
| | (T3A, E15K, E32H, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 95 | IL-2-IL-15 Chimera 9 | APTSSSTKKTQLQLKHLLLDLQMILNGIQSMHIDAT |
| | (E15K, E32H, S87D, C125S) | TFKFYMPKKATELKHLQCLEEELKPLEEVLNLAQS |
| | | KNFHLRPRDLIDNINVIVLELKGSETTFMCEYADET |
| | | ATIVEFLNRWITFSQSIISTLT |
| 96 | IL-2-IL-15 Chimera 10 | APASSSTKKTQLQLKHLLLDLQMILNGIQSMHIDAT |
| | (T3A, E15K, E32H, S87D, | TFKFYMPKKATELKHLQCLEEELKPLEEVLNLAQS |
| | C125S) | KNFHLRPRDLIDNINVIVLELKGSETTFMCEYADET |
| | | ATIVEFLNRWITFSQSIISTLT |
| 97 | IL-2-IL-15 Chimera 11 | APTSSSTKKTQLQLEELLLDLQMILNGIQSMEIDAT |
| | (H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 98 | IL-2-IL-15 Chimera 12 | APASSSTKKTQLQLEELLLDLQMILNGIQSMEIDAT |
| | (T3A, H16E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 99 | IL-2-IL-15 Chimera 13 | APTSSSTKKTQLQLEFLLLDLQMILNGIQSMEIDAT |
| | (H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 100 | IL-2-IL-15 Chimera 14 | APASSSTKKTQLQLEFLLLDLQMILNGIQSMEIDAT |
| | (T3A, H16F, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 101 | IL-2-IL-15 Chimera 15 | APTSSSTKKTQLQLEILLLDLQMILNGIQSMEIDATT |
| | (H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 102 | IL-2-IL-15 Chimera 16 | APASSSTKKTQLQLEILLLDLQMILNGIQSMEIDATT |
| | (T3A, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 103 | IL-2-IL-15 Chimera 17 | APTSSSTKKTQLQLEVLLLDLQMILNGIQSMEIDAT |
| | (H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 104 | IL-2-IL-15 Chimera 18 | APASSSTKKTQLQLEVLLLDLQMILNGIQSMEIDAT |
| | (T3A, H16V, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 105 | IL-2-IL-15 Chimera 19 | APTSSSTKKTQLQLEHLLLTLQMILNGIQSMEIDAT |
| | (D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 106 | IL-2-IL-15 Chimera 20 | APASSSTKKTQLQLEHLLLTLQMILNGIQSMEIDAT |
| | (T3A, D20T, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 107 | IL-2-IL-15 Chimera 21 | APTSSSTKKTQLQLEHLLLALQMILNGIQSMEIDAT |
| | (D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 108 | IL-2-IL-15 Chimera 22 | APASSSTKKTQLQLEHLLLALQMILNGIQSMEIDAT |
| | (T3A, D20A, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 109 | IL-2-IL-15 Chimera 23 | APTSSSTKKTQLQLEHLLLELQMILNGIQSMEIDAT |
| | (D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 110 | IL-2-IL-15 Chimera 24 | APASSSTKKTQLQLEHLLLELQMILNGIQSMEIDAT |
| | (T3A, D20E, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 111 | IL-2-IL-15 Chimera 25 | APTSSSTKKTQLQLEHLLLKLQMILNGIQSMEIDAT |
| | (D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 112 | IL-2-IL-15 Chimera 26 | APASSSTKKTQLQLEHLLLKLQMILNGIQSMEIDAT |
| | (T3A, D20K, C125S) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIISTLT |
| 113 | IL-2-IL-15 Chimera 27 | APTSSSTKKTQLQLEHLLLDLQMILNGIQSMEIDAT |
| | (N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 114 | IL-2-IL-15 Chimera 28 | APASSSTKKTQLQLEHLLLDLQMILNGIQSMEIDAT |
| | (T3A, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 115 | IL-2-IL-15 Chimera 29 | APTSSSTKKTQLQLEHLLLDLQMILNGIQSMEIDAT |
| | (192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 116 | IL-2-IL-15 Chimera 30 | APASSSTKKTQLQLEHLLLDLQMILNGIQSMEIDAT |
| | (T3A, I92A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 117 | IL-2-IL-15 Chimera 31 | APTSSSTKKTQLQLEHLLLDLQMILNGIQSMEIDAT |
| | (192G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 118 | IL-2-IL-15 Chimcra 32 | APASSSTKKTQLQLEHLLLDLQMILNGIQSMEIDAT |
| | (T3A, 192G, C125S) | TFKFYMPKKATELKHLQCLEEELKPLEEVLNLAQS |
| | | KNFHLRPRDLISNINVGVLELKGSETTFMCEYADET |
| | | ATIVEFLNRWITFSQSIISTLT |
| 119 | IL-2-IL-15 Chimera 33 | APTSSSTKKTQLQLEHLLLDLQMILNGIQSMEIDAT |
| | (192D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 120 | IL-2-IL-15 Chimera 34 | APASSSTKKTQLQLEHLLLDLQMILNGIQSMEIDAT |
| | (T3A, I92D, C125S) | TFKFYMPKKATELKHLQCLEEELKPLEEVLNLAQS |
| | | KNFHLRPRDLISNINVDVLELKGSETTFMCEYADET |
| | | ATIVEFLNRWITFSQSIISTLT |
| 121 | IL-2-IL-15 Chimera 35 | APTSSSTKKTQLQLEHLLLDLQMILNGIQSMEIDAT |
| | (192E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 122 | IL-2-IL-15 Chimera 36 | APASSSTKKTQLQLEHLLLDLQMILNGIQSMEIDAT |
| | (T3A, I92E, C125S) | TFKFYMPKKATELKHLQCLEEELKPLEEVLNLAQS |
| | | KNFHLRPRDLISNINVEVLELKGSETTFMCEYADET |
| | | ATIVEFLNRWITFSQSIISTLT |
| 123 | IL-2-IL-15 Chimera 37 | APTSSSTKKTQLQLEELLLDLQMILNGIQSMEIDAT |
| | (H16E, I92A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 124 | IL-2-IL-15 Chimera 38 | APASSSTKKTQLQLEELLLDLQMILNGIQSMEIDAT |
| | (T3A, H16E, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 125 | IL-2-IL-15 Chimera 39 | APTSSSTKKTQLQLKHLLLDLQMILNGIQSMEIDAT |
| | (E15K, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 126 | IL-2-IL-15 Chimera 40 | APASSSTKKTQLQLKHLLLDLQMILNGIQSMEIDAT |
| | (T3A, E15K, 192A, C125S) | TFKFYMPKKATELKHLQCLEEELKPLEEVLNLAQS |
| | | KNFHLRPRDLISNINVAVLELKGSETTFMCEYADET |
| | | ATIVEFLNRWITFSQSIISTLT |
| 127 | IL-2-IL-15 Chimera 41 | APTSSSTKKTQLQLKILLLDLQMILNGIQSMEIDATT |
| | (E15K, H16I, C125S) | FKFYMPKKATELKHLQCLEEELKPLEEVLNLAQSK |
| | | NFHLRPRDLISNINVIVLELKGSETTFMCEYADETA |
| | | TIVEFLNRWITFSQSIISTLT |
| 128 | IL-2-IL-15 Chimera 42 | APTSSSTKKTQLQLKHLLLDLQMILNGIQSMEIDAT |
| | (E15K, N88A, C125S) | TFKFYMPKKATELKHLQCLEEELKPLEEVLNLAQS |
| | | KNFHLRPRDLISAINVIVLELKGSETTFMCEYADET |
| | | ATIVEFLNRWITFSQSIISTLT |
| 129 | IL-2-IL-15 Chimcra 43 | APTSSSTKKTQLQLEHLCLDLQMILNGIQSMEIDAT |
| | (L18C) | TFKFYMPKKATELKHLQCLEEELKPLEEVLNLAQS |
| | | KNFHLRPRDLISNINVIVLELKGSETTFMCEYADET |
| | | ATIVEFLNRWITFCQSIISTLT |
| 130 | IL-2-IL-15 Chimera 44 | APASSSTKKTQLQLEHLCLDLQMILNGIQSMEIDAT |
| | (T3A, L18C) | TFKFYMPKKATELKHLQCLEEELKPLEEVLNLAQS |
| | | KNFHLRPRDLISNINVIVLELKGSETTFMCEYADET |
| | | ATIVEFLNRWITFCQSIISTLT |
| 131 | IL-2-IL-15 Chimera 45 | APTSSSTKKTQLQLEHLLLDLQMILNGIQSMEIDAT |
| | (C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |
| 132 | IL-2-IL-15 Chimera 46 | APASSSTKKTQLQLEHLLLDLQMILNGIQSMEIDAT |
| | (T3A, C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |
| 133 | IL-2-IL-15 Chimera 47 | APTSSSTKKTQLQLEHLLLDLQMILNGIQSMEIDAT |
| | (C125S, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFSKSIISTLT |
| 134 | IL-2-IL-15 Chimera 48 | APASSSTKKTQLQLEHLLLDLQMILNGIQSMEIDAT |
| | (T3A, C125S, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFSKSIISTLT |
| 135 | IL-2-IL-15 Chimera 49 | APTSSSTKKTQLQLEHLLLDLQMILNGIQSMEIDAT |
| | (C125S, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFSRSIISTLT |
| 136 | IL-2-IL-15 Chimera 50 | APASSSTKKTQLQLEHLLLDLQMILNGIQSMEIDAT |
| | (T3A, C125S, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFSRSIISTLT |
| 137 | IL-2-IL-15 Chimera 51 | APTSSSTKKTQLQLEHLLLDLQMILNGIQSMEIDAT |
| | (C125S, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFSSSIISTLT |
| 138 | IL-2-IL-15 Chimera 52 | APASSSTKKTQLQLEHLLLDLQMILNGIQSMEIDAT |
| | (T3A, C125S, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFSSSIISTLT |
| 139 | IL-2-IL-15 Chimera 53 | APTSSSTKKTQLQLEHLLLDLQMILNGIQSMEIDAT |
| | (C125S, Q126T) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIISTLT |
| 140 | IL-2-IL-15 Chimera 54 | APASSSTKKTQLQLEHLLLDLQMILNGIQSMEIDAT |
| | (T3A, C125S, Q126T) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIISTLT |
| 141 | IL-2-IL-15 Chimera 55 | APTSSSTKKTQLQLEHLLLDLQMILNGIQSMEIDAT |
| | (C125S, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIIRTLT |
| 142 | IL-2-IL-15 Chimera 56 | APASSSTKKTQLQLEHLLLDLQMILNGIQSMEIDAT |
| | (T3A, C125S, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSQSIIRTLT |
| 143 | IL-2-IL-15 Chimera 57 | APTSSSTKKTQLQLEHLCLDLQMILNGIQSMEIDAT |
| | (L18C, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFCESIISTLT |
| 144 | IL-2-IL-15 Chimera 58 | APASSSTKKTQLQLEHLCLDLQMILNGIQSMEIDAT |
| | (T3A, L18C, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFCESIISTLT |
| 145 | IL-2-IL-15 Chimera 59 | APTSSSTKKTQLQLEHLCLDLQMILNGIQSMEIDAT |
| | (L18C, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFCKSIISTLT |
| 146 | IL-2-IL-15 Chimera 60 | APASSSTKKTQLQLEHLCLDLQMILNGIQSMEIDAT |
| | (T3A, L18C, Q126K) | TFKFYMPKKATELKHLQCLEEELKPLEEVLNLAQS |
| | | KNFHLRPRDLISNINVIVLELKGSETTFMCEYADET |
| | | ATIVEFLNRWITFCKSIISTLT |
| 147 | IL-2-IL-15 Chimera 61 | APTSSSTKKTQLQLEHLCLDLQMILNGIQSMEIDAT |
| | (L18C, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFCRSIISTLT |
| 148 | IL-2-IL-15 Chimera 62 | APASSSTKKTQLQLEHLCLDLQMILNGIQSMEIDAT |
| | (T3A, L18C, Q126R) | TFKFYMPKKATELKHLQCLEEELKPLEEVLNLAQS |
| | | KNFHLRPRDLISNINVIVLELKGSETTFMCEYADET |
| | | ATIVEFLNRWITFCRSIISTLT |
| 149 | IL-2-IL-15 Chimera 63 | APTSSSTKKTQLQLEHLCLDLQMILNGIQSMEIDAT |
| | (L18C, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFCSSIISTLT |
| 150 | IL-2-IL-15 Chimera 64 | APASSSTKKTQLQLEHLCLDLQMILNGIQSMEIDAT |
| | (T3A, L18C, Q126S) | TFKFYMPKKATELKHLQCLEEELKPLEEVLNLAQS |
| | | KNFHLRPRDLISNINVIVLELKGSETTFMCEYADET |
| | | ATIVEFLNRWITFCSSIISTLT |
| 151 | IL-2-IL-15 Chimera 65 | APTSSSTKKTQLQLEHLLLDLQMILNGIQSMEIDAT |
| | (192A, C125S, Q126E) | TFKFYMPKKATELKHLQCLEEELKPLEEVLNLAQS |
| | | KNFHLRPRDLISNINVAVLELKGSETTFMCEYADET |
| | | ATIVEFLNRWITFSESIISTLT |
| 152 | IL-2-IL-15 Chimera 66 | APASSSTKKTQLQLEHLLLDLQMILNGIQSMEIDAT |
| | (T3A, I92A, C125S, Q126E) | TFKFYMPKKATELKHLQCLEEELKPLEEVLNLAQS |
| | | KNFHLRPRDLISNINVAVLELKGSETTFMCEYADET |
| | | ATIVEFLNRWITFSESIISTLT |
| 153 | IL-2-IL-15 Chimera 67 | APTSSSTKKTQLQLEHLLLDLQMILNGIQSMEIDAT |
| | (C125S, Q126T, S130R) | TFKFYMPKKATELKHLQCLEEELKPLEEVLNLAQS |
| | | KNFHLRPRDLISNINVIVLELKGSETTFMCEYADET |
| | | ATIVEFLNRWITFSTSIIRTLT |
| 154 | IL-2-IL-15 Chimera 68 | APASSSTKKTQLQLEHLLLDLQMILNGIQSMEIDAT |
| | (T3A, C125S, Q126T, | TFKFYMPKKATELKHLQCLEEELKPLEEVLNLAQS |
| | S130R) | KNFHLRPRDLISNINVIVLELKGSETTFMCEYADET |
| | | ATIVEFLNRWITFSTSIIRTLT |
| 155 | IL-2-IL-15 Chimera 69 | APASSSTKKTQLQLEHLSLDLQMILNGIQSMEIDAT |
| | (T3A, L18S, C125S, Q126E) | TFKFYMPKKATELKHLQCLEEELKPLEEVLNLAQS |
| | | KNFHLRPRDLISNINVIVLELKGSETTFMCEYADET |
| | | ATIVEFLNRWITFSESIISTLT |
| 156 | Mouse IL-2-IL-15 Chimera | APTSSSTSSSTAEAQQQQQQQQQHLEQLLMDL |
| | | QELLSRMQSMEIDATTFKFYLPKQATELKDLQCLE |
| | | DELGPLRHVLDLTQSKSFQLEDAENFISNIRVTVVK |
| | | LKGSDNTFECQFDDESATVVDFLRRWIAFSQSIISTS |
| | | PQ |
| 157 | hIL-2Rα | ELCDDDPPEIPHATFKAMAYKEGTMLNCECKRGFR |
| | (extracellular domain) | RIKSGSLYMLCTGNSSHSSWDNQCQCTSSATRNTT |
| | | KQVTPQPEEQKERKTTEMQSPMQPVDQASLPGHC |
| | | REPPPWENEATERIYHFVVGQMVYYQCVQGYRAL |
| | | HRGPAESVCKMTHGKTRWTQPQLICTGEMETSQFP |
| | | GEEKPQASPEGRPESETSCLVTTTDFQIQTEMAATM |
| | | ETSIFTTEYQ |
| 158 | hIL-2Rβ | AVNGTSQFTCFYNSRANISCVWSQDGALQDTSCQV |
| | (extracellular domain) | HAWPDRRRWNQTCELLPVSQASWACNLILGAPDS |
| | | QKLTTVDIVTLRVLCREGVRWRVMAIQDFKPFENL |
| | | RLMAPISLQVVHVETHRCNISWEISQASHYFERHLE |
| | | FEARTLSPGHTWEEAPLLTLKQKQEWICLETLTPDT |
| | | QYEFQVRVKPLQGEFTTWSPWSQPLAFRTKPAALG |
| | | KD |
| 159 | hIL-2Rγ | LNTTILTPNGNEDTTADFFLTTMPTDSLSVSTLPLPE |
| | (extracellular domain) | VQCFVFNVEYMNCTWNSSSEPQPTNLTLHYWYKN |
| | | SDNDKVQKCSHYLFSEEITSGCQLQKKEIHLYQTFV |
| | | VQLQDPREPRRQATQMLKLQNLVIPWAPENLTLH |
| | | KLSESQLELNWNNRFLNHCLEHLVQYRTDWDHSW |
| | | TEQSVDYRHKFSLPSVDGQKRYTFRVRSRFNPLCG |
| | | SAQHWSEWSHPIHWGSNTSKENPFLFALEA |
| 160 | Anti-hPD-1- IL-2-IL-15 | QVQLLESGGGLVKPGGSLRLSCAASGRTYAMGWF |
| | Chimera 69 (T3A, L18S, | RRAPGKESEFVAEIRWSSTFTDYADSVKGRFTISRD |
| | C125S, Q126E) (Hole) | NAENSAYLQMNSLRAEDTAVYHCAAQTSQRGTLY |
| | | DSYGQGTQVTVSSESKYGPPCPPCPAPEFLGGPSVF |
| | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVCTLPPSQEEMTKNQVSLSCAVKGFYPSDIAVE |
| | | WESNGQPENNYKTTPPVLDSDGSFFLVSRLTVDKS |
| | | RWQEGNVFSCSVMHEALHNHYTQKSLSLSLGAGG |
| | | GGSGGGGSGGGGSAPASSSTKKTQLQLEHLSLDLQ |
| | | MILNGIQSMEIDATTFKFYMPKKATELKHLQCLEEE |
| | | LKPLEEVLNLAQSKNFHLRPRDLISNINVIVLELKGS |
| | | ETTFMCEYADETATIVEFLNRWITFSESIISTLT |
| 161 | Anti-hPD-1 (Knob) | QVQLLESGGGLVKPGGSLRLSCAASGRTYAMGWF |
| | | RRAPGKESEFVAEIRWSSTFTDYADSVKGRFTISRD |
| | | NAENSAYLQMNSLRAEDTAVYHCAAQTSQRGTLY |
| | | DSYGQGTQVTVSSESKYGPPCPPCPAPEFLGGPSVF |
| | | LFPPKPKDTLMISRTPEVTCVVVDVSQEDPEVQFN |
| | | WYVDGVEVHNAKTKPREEQFNSTYRVVSVLTVLH |
| | | QDWLNGKEYKCKVSNKGLPSSIEKTISKAKGQPRE |
| | | PQVYTLPPCQEEMTKNQVSLWCLVKGFYPSDIAVE |
| | | WESNGQPENNYKTTPPVLDSDGSFFLYSRLTVDKS |
| | | RWQEGNVFSCSVMHEALHNHYTQKSLSLSLGK |
| 162 | hIgGl Fc | DKTHTCPPCPAPELLGGPSVFLFPPKPKDTLMISRTP |
| | | EVTCVVVDVSHEDPEVKFNWYVDGVEVHNAKTK |
| | | PREEQYNSTYRVVSVLTVLHQDWLNGKEYKCKVS |
| | | NKALPAPIEKTISKAKGQPREPQVYTLPPSREEMTK |
| | | NQVSLTCLVKGFYPSDIAVEWESNGQPENNYKTTP |
| | | PVLDSDGSFFLYSKLTVDKSRWQQGNVFSCSVMH |
| | | EALHNHYTQKSLSLSPGK |
| 163 | hIgG1 Fc Mutein | DKTHTCPPCPAPELLGGPSVFLFPPKPKDTLMISRTP |
| | (N297A) | EVTCVVVDVSHEDPEVKFNWYVDGVEVHNAKTK |
| | | PREEQYASTYRVVSVLTVLHQDWLNGKEYKCKVS |
| | | NKALPAPIEKTISKAKGQPREPQVYTLPPSREEMTK |
| | | NQVSLTCLVKGFYPSDIAVEWESNGQPENNYKTTP |
| | | PVLDSDGSFFLYSKLTVDKSRWQQGNVFSCSVMH |
| | | EALHNHYTQKSLSLSPGK |
| 164 | hIgG1 Fc Mutein | DKTHTCPPCPAPEAAGGPSVFLFPPKPKDTLMISRT |
| | (LALA, N297A) | PEVTCVVVDVSHEDPEVKFNWYVDGVEVHNAKT |
| | | KPREEQYASTYRVVSVLTVLHQDWLNGKEYKCKV |
| | | SNKALPAPIEKTISKAKGQPREPQVYTLPPSREEMT |
| | | KNQVSLTCLVKGFYPSDIAVEWESNGQPENNYKTT |
| | | PPVLDSDGSFFLYSKLTVDKSRWQQGNVFSCSVMH |
| | | EALHNHYTQKSLSLSPGK |
| 165 | hIgG1 Fc Mutein | DKTHTCPPCPAPELLGGPSVFLFPPKPKDTLMISRTP |
| | (S354C, T366W) | EVTCVVVDVSHEDPEVKFNWYVDGVEVHNAKTK |
| | | PREEQYNSTYRVVSVLTVLHQDWLNGKEYKCKVS |
| | | NKALPAPIEKTISKAKGQPREPQVYTLPPCREEMTK |
| | | NQVSLWCLVKGFYPSDIAVEWESNGQPENNYKTT |
| | | PPVLDSDGSFFLYSKLTVDKSRWQQGNVFSCSVMH |
| | | EALHNHYTQKSLSLSPGK |
| 166 | hIgG1 Fc Mutein | DKTHTCPPCPAPELLGGPSVFLFPPKPKDTLMISRTP |
| | (Y349C, T366S, L368A, | EVTCVVVDVSHEDPEVKFNWYVDGVEVHNAKTK |
| | Y407V) | PREEQYNSTYRVVSVLTVLHQDWLNGKEYKCKVS |
| | | NKALPAPIEKTISKAKGQPREPQVCTLPPSREEMTK |
| | | NQVSLSCAVKGFYPSDIAVEWESNGQPENNYKTTP |
| | | PVLDSDGSFFLVSKLTVDKSRWQQGNVFSCSVMH |
| | | EALHNHYTQKSLSLSPGK |
| 167 | hIgG1 Fc Mutein | DKTHTCPPCPAPELLGGPSVFLFPPKPKDTLMISRTP |
| | (S354C, T366W, N297A) | EVTCVVVDVSHEDPEVKFNWYVDGVEVHNAKTK |
| | | PREEQYASTYRVVSVLTVLHQDWLNGKEYKCKVS |
| | | NKALPAPIEKTISKAKGQPREPQVYTLPPCREEMTK |
| | | NQVSLWCLVKGFYPSDIAVEWESNGQPENNYKTT |
| | | PPVLDSDGSFFLYSKLTVDKSRWQQGNVFSCSVMH |
| | | EALHNHYTQKSLSLSPGK |
| 168 | hIgG1 Fc Mutein | DKTHTCPPCPAPELLGGPSVFLFPPKPKDTLMISRTP |
| | (Y349C, T366S, L368A, | EVTCVVVDVSHEDPEVKFNWYVDGVEVHNAKTK |
| | Y407V, N297A) | PREEQYASTYRVVSVLTVLHQDWLNGKEYKCKVS |
| | | NKALPAPIEKTISKAKGQPREPQVCTLPPSREEMTK |
| | | NQVSLSCAVKGFYPSDIAVEWESNGQPENNYKTTP |
| | | PVLDSDGSFFLVSKLTVDKSRWQQGNVFSCSVMH |
| | | EALHNHYTQKSLSLSPGK |
| 169 | hIgG1 Fc Mutein | DKTHTCPPCPAPEAAGGPSVFLFPPKPKDTLMISRT |
| | (S354C, T366W, LALA, | PEVTCVVVDVSHEDPEVKFNWYVDGVEVHNAKT |
| | N297A) | KPREEQYASTYRVVSVLTVLHQDWLNGKEYKCKV |
| | | SNKALPAPIEKTISKAKGQPREPQVYTLPPCREEMT |
| | | KNQVSLWCLVKGFYPSDIAVEWESNGQPENNYKT |
| | | TPPVLDSDGSFFLYSKLTVDKSRWQQGNVFSCSVM |
| | | HEALHNHYTQKSLSLSPGK |
| 170 | hIgG1 Fc Mutein | DKTHTCPPCPAPEAAGGPSVFLFPPKPKDTLMISRT |
| | (Y349C, T366S, L368A, | PEVTCVVVDVSHEDPEVKFNWYVDGVEVHNAKT |
| | Y407V, LALA, N297A) | KPREEQYASTYRVVSVLTVLHQDWLNGKEYKCKV |
| | | SNKALPAPIEKTISKAKGQPREPQVCTLPPSREEMT |
| | | KNQVSLSCAVKGFYPSDIAVEWESNGQPENNYKTT |
| | | PPVLDSDGSFFLVSKLTVDKSRWQQGNVFSCSVMH |
| | | EALHNHYTQKSLSLSPGK |
| 171 | hIgG2 Fc | ERKCCVECPPCPAPPVAGPSVFLFPPKPKDTLMISR |
| | | TPEVTCVVVDVSHEDPEVQFNWYVDGVEVHNAKT |
| | | KPREEQFNSTFRVVSVLTVVHQDWLNGKEYKCKV |
| | | SNKGLPAPIEKTISKTKGQPREPQVYTLPPSREEMT |
| | | KNQVSLTCLVKGFYPSDIAVEWESNGQPENNYKTT |
| | | PPMLDSDGSFFLYSKLTVDKSRWQQGNVFSCSVM |
| | | HEALHNHYTQKSLSLSPGK |
| 172 | hIgG4 Fc | ESKYGPPCPPCPAPEFLGGPSVFLFPPKPKDTLMISR |
| | | TPEVTCVVVDVSQEDPEVQFNWYVDGVEVHNAKT |
| | | KPREEQFNSTYRVVSVLTVLHQDWLNGKEYKCKV |
| | | SNKGLPSSIEKTISKAKGQPREPQVYTLPPSQEEMT |
| | | KNQVSLTCLVKGFYPSDIAVEWESNGQPENNYKTT |
| | | PPVLDSDGSFFLYSRLTVDKSRWQEGNVFSCSVMH |
| | | EALHNHYTQKSLSLSLGK |
| 173 | hIgG4 Fc (Hole) | ESKYGPPCPPCPAPEFLGGPSVFLFPPKPKDTLMISR |
| | | TPEVTCVVVDVSQEDPEVQFNWYVDGVEVHNAKT |
| | | KPREEQFNSTYRVVSVLTVLHQDWLNGKEYKCKV |
| | | SNKGLPSSIEKTISKAKGQPREPQVCTLPPSQEEMT |
| | | KNQVSLSCAVKGFYPSDIAVEWESNGQPENNYKTT |
| | | PPVLDSDGSFFLVSRLTVDKSRWQEGNVFSCSVMH |
| | | EALHNHYTQKSLSLSLGA |
| 174 | hIgG4 Fc (Knob) | ESKYGPPCPPCPAPEFLGGPSVFLFPPKPKDTLMISR |
| | | TPEVTCVVVDVSQEDPEVQFNWYVDGVEVHNAKT |
| | | KPREEQFNSTYRVVSVLTVLHQDWLNGKEYKCKV |
| | | SNKGLPSSIEKTISKAKGQPREPQVYTLPPCQEEMT |
| | | KNQVSLWCLVKGFYPSDIAVEWESNGQPENNYKT |
| | | TPPVLDSDGSFFLYSRLTVDKSRWQEGNVFSCSVM |
| | | HEALHNHYTQKSLSLSLGK |
| 175 | hIgG4 Fc (Hole) | ESKYGPPCPPCPAPEFLGGPSVFLFPPKPKDTLMISR |
| | | TPEVTCVVVDVSQEDPEVQFNWYVDGVEVHNAKT |
| | | KPREEQFNSTYRVVSVLTVLHQDWLNGKEYKCKV |
| | | SNKGLPSSIEKTISKAKGQPREPQVCTLPPSQEEMT |
| | | KNQVSLSCAVKGFYPSDIAVEWESNGQPENNYKTT |
| | | PPVLDSDGSFFLVSRLTVDKSRWQEGNVFSCSVMH |
| | | EALHNHYTQKSLSLSLGK |

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

What is claimed is:

1. A fusion protein comprising an IL-2 domain, an anti-PD-1 single domain antibody (sdAb), and a fragment crystallizable (Fe) domain; wherein the anti-PD-1 sdAb comprises a CDR1 of SEQ ID NO: 1, a CDR2 of SEQ ID NO: 2, and a CDR3 of SEQ ID NO: 3.

2. The fusion protein of claim 1, comprising an IL-2 domain, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the IL-2 domain directly or via a peptide linker; and wherein the C-terminus of the second anti-PD-1 sdAb is connected to the N-terminus of the second peptide chain of the Fc domain directly or via a peptide linker.

3. The fusion protein of claim 1, comprising first and second IL-2 domains, first and second anti-PD-1 sdAbs, and an Fc domain comprising first and second peptide chains; wherein the C-terminus of the first anti-PD-1 sdAb is connected to the N-terminus of the first peptide chain of the Fc domain directly or via a peptide linker, and the C-terminus of the first peptide chain of the Fc domain is connected to the N-terminus of the first IL-2 domain directly or via a peptide linker; and wherein the C-terminus of the second anti-PD-1 sdAb is connected to the N-terminus of the second peptide chain of the Fc domain directly or via a peptide linker, and the C-terminus of the second peptide chain of the Fc domain is connected to the N-terminus of the second IL-2 domain directly or via a peptide linker.

4. The fusion protein of any one of claims 1 to 3, wherein the anti-PD-1 sdAb is an anti-PD-1 V_{H}H sdAb.

5. The fusion protein of any one of claims 1 to 4, wherein each anti-PD-1 sdAb independently comprises the amino acid sequence of SEQ ID NO: 4 or 5.

6. The fusion protein of any one of claims 1 to 5, wherein each anti-PD-1 sdAb comprises the amino acid sequence of SEQ ID NO: 4.

7. The fusion protein of any one of claims 1 to 5, wherein each anti-PD-1 sdAb comprises the amino acid sequence of SEQ ID NO: 5.

8. The fusion protein of any one of claims 1 to 7, wherein each anti-PD-1 sdAb is independently a human or humanized anti-PD-1 sdAb.

9. The fusion protein of any one of claims 1 to 8, wherein the Fc domain is a hIgG1 Fc domain, a hIgG2 Fc domain, a hIgG4 Fc domain, or a mutein thereof.

10. The fusion protein of any one of claims 1 to 9, wherein the Fc domain is a hIgG4 Fc domain or a mutein thereof.

11. The fusion protein of any one of claims 1 to 10, wherein the Fc domain comprises the amino acid sequences of SEQ ID NO: 173, 174, or 175.

12. The fusion protein of any one of claims 1 to 11, wherein the Fc domain comprises the amino acid sequences of SEQ ID NOs: 173 and 174, or 174 and 175.

13. The fusion protein of any one of claims 1 to 12, wherein the Fc domain is in a knobs-in-holes configuration.

14. The fusion protein of any one of claims 1 to 13, wherein each IL-2 domain is independently an IL-2 mutein comprising: (i) an amino acid substitution at position L18, Q126, or S130; or (ii) a replacement of the amino acid residues from positions N29 to L40 with an IL-15 hinge fragment-containing peptide; a disulfide bond formed between two amino acid residues from positions N30 to L80; or an amino acid substitution at position E15, H16, D20, K32, R38, M39, L40, T41, F42, K43, F44, Y45, K76, S87, N88, or 192.

15. The fusion protein of any one of claims 1 to 14, wherein each IL-2 domain is independently an IL-2 mutein comprising an amino acid substitution at position L18.

16. The fusion protein of any one of claims 1 to 15, wherein each IL-2 domain is independently an IL-2 mutein comprising an amino acid substitution of L18C or L18S.

17. The fusion protein of any one of claims 1 to 16, wherein each IL-2 domain is independently an IL-2 mutein comprising an amino acid substitution of L18S.

18. The fusion protein of any one of claims 1 to 17, wherein each IL-2 domain is independently an IL-2 mutein comprising an amino acid substitution at position Q126.

19. The fusion protein of any one of claims 1 to 18, wherein each IL-2 domain is independently an IL-2 mutein comprising an amino acid substitution of Q126E, Q126K, Q126R, Q126S, or Q126T.

20. The fusion protein of any one of claims 1 to 19, wherein each IL-2 domain is independently an IL-2 mutein comprising an amino acid substitution of Q126E.

21. The fusion protein of any one of claims 1 to 20, wherein each IL-2 domain is independently an IL-2 mutein comprising an amino acid substitution at position S130.

22. The fusion protein of any one of claims 1 to 21, wherein each IL-2 domain is independently an IL-2 mutein comprising an amino acid substitution of S130A, S130E, S130Q, or S130R.

23. The fusion protein of any one of claims 1 to 22, wherein each IL-2 domain is independently an IL-2 mutein comprising amino acid substitution of (i) Q126E or Q126T; (ii) L18C, and Q126E, Q126K, Q126R, or Q126S; (iii) L18S and Q126E; (iv) Q126T and S130R; or (v) L18C, Q126E, and S130E.

24. The fusion protein of any one of claims 1 to 23, wherein each IL-2 domain is independently an IL-2 mutein comprising amino acid substitutions of L18S and Q126E.

25. The fusion protein of any one of claims 1 to 24, wherein each IL-2 domain is independently an IL-2 mutein comprising a replacement of the amino acid residues from positions N29 to L40 with an IL-15 hinge fragment-containing peptide.

26. The fusion protein of any one of claims 1 to 25, wherein each IL-2 domain is independently an IL-2 mutein comprising a replacement of the amino acid residues from positions N29 to L40 having the amino acid sequence of SEQ ID NO: 84 with an IL-15 hinge fragment-containing peptide having the amino acid sequence of SEQ ID NO: 85 or 86.

27. The fusion protein of any one of claims 1 to 26, wherein each IL-2 domain is independently an IL-2 mutein comprising (i) amino acid substitutions of L18S and Q126E and (ii) a replacement of the amino acid residues from positions N29 to L40 having the amino acid sequence of SEQ ID NO: 84 with an IL-15 hinge fragment-containing peptide having the amino acid sequence of SEQ ID NO: 85 or 86.

28. The fusion protein of any one of claims 1 to 27, wherein each IL-2 domain is independently an IL-2 mutein comprising (i) amino acid substitutions of L18S and Q126E and (ii) a replacement of the amino acid residues from positions N29 to L40 having the amino acid sequence of SEQ ID NO: 84 with an IL-15 hinge fragment-containing peptide having the amino acid sequence of SEQ ID NO: 85.

29. The fusion protein of any one of claims 1 to 27, wherein each IL-2 domain is independently an IL-2 mutein comprising (i) amino acid substitutions of L18S and Q126E and (ii) a replacement of the amino acid residues from positions N29 to L40 having the amino acid sequence of SEQ ID NO: 84 with an IL-15 hinge fragment-containing peptide having the amino acid sequence of SEQ ID NO: 86.

30. The fusion protein of any one of claims 1 to 13, wherein each IL-2 domain independently comprises the amino acid sequence of any one of SEQ ID NOs: 46 to 83 and 87 to 156.

31. The fusion protein of any one of claims 1 to 13 and 30, wherein each IL-2 domain is independently an IL-2 mutein comprising the amino acid sequence of any one of SEQ ID NOs: 52 to 83 and 87 to 155.

32. The fusion protein of any one of claims 1 to 13, 30, and 31, wherein the IL-2 domain comprises the amino acid sequence of any one of SEQ ID NOs: 87 to 155.

33. The fusion protein of any one of claims 1 to 12 and 30 to 32, wherein the IL-2 domain comprises the amino acid sequence of SEQ ID NO: 155.

34. The fusion protein of any one of claims 1 to 33, wherein each peptide linker independently comprises the amino acid sequence of GSG or any one of SEQ ID NOs: 6 to 45.

35. The fusion protein of any one of claims 1 to 34, wherein each peptide linker independently comprises the amino acid sequence of SEQ ID NO: 13, 14, 15, or 16.

36. The fusion protein of any one of claims 1 to 35, wherein each peptide linker comprises the amino acid sequence of SEQ ID NO: 15.

37. The fusion protein of claim 1, wherein the fusion protein comprises the amino acid sequences of SEQ ID NOs: 160 and 161.

38. A pharmaceutical composition comprising the fusion protein of any one of claims 1 to 37 and a pharmaceutically acceptable excipient.

39. The pharmaceutical composition of claim 38, wherein the pharmaceutical composition is in single dosage form.

40. The pharmaceutical composition of claim 38 or 39, wherein the pharmaceutical composition is a solid.

41. The pharmaceutical composition of claim 38 or 39, wherein the pharmaceutical composition is in a parenteral dosage form.

42. The pharmaceutical composition of claim 41, the pharmaceutical composition is in an intravenous dosage form.

43. The pharmaceutical composition of any one of claims 38, 39, 41, and 42, wherein the pharmaceutical composition is a solution.

44. A method of treating one or more symptoms of treating, preventing, or ameliorating one or more symptoms of a proliferative disease in a subject, comprising administering to the subject in need thereof a therapeutically effective amount of the fusion protein of any one of claims 1 to 37 or the pharmaceutical composition of any one of claims 38 to 43.

45. The method of claim 44, wherein the proliferative disease is cancer.

46. The method of claim 44 or 45, wherein the proliferative disease is metastatic cancer.

47. A method of inhibiting the growth of a cell, comprising contacting the cell with an effective amount of the fusion protein of any one of claims 1 to 37 or the pharmaceutical composition of any one of claims 38 to 43.

48. The method of claim 47, wherein the cell is a cancerous cell.


SEQUENCE LISTING (175 sequences)

SEQ ID NO: 1 - protein, 5 aa, synthetic construct
  REGION 1..5: note = Anti-hPD-1-VHH CDR1 (IMGT)
GRTYA

SEQ ID NO: 2 - protein, 8 aa, synthetic construct
  REGION 1..8: note = Anti-hPD-1-VHH CDR2 (IMGT)
IRWSSTFT

SEQ ID NO: 3 - protein, 12 aa, synthetic construct
  REGION 1..12: note = Anti-hPD-1-VHH CDR3 (IMGT)
AAQTSQRGTLYD

SEQ ID NO: 4 - protein, 117 aa, synthetic construct
  REGION 1..117: note = Anti-hPD-1-VHH-A
QVQLLESGGGLVKPGGSLRLSCAASGRTYAMGWFRRAPGKESEFVAEIRWSSTFTDYADSVKGRFTISRDNAENSAYLQMNSLRAEDTAVYHCAAQTSQRGTLYDSYGQGTQVTVSS

SEQ ID NO: 5 - protein, 117 aa, synthetic construct
  REGION 1..117: note = Anti-hPD-1-VHH-B
QVQLVESGGGVVQPGGSLRLSCAASGRTYAMGWFRQAPGKESEFVAEIRWSGSFTYYADSVKGRFTISRDNSENTAYLQMNSLRAEDTAVYRCAAQTSQRGTLYDHYGQGTLVTVSS

SEQ ID NO: 6 - protein, 6 aa, synthetic construct
  REGION 1..6: note = (GSG)2 Linker
GSGGSG

SEQ ID NO: 7 - protein, 9 aa, synthetic construct
  REGION 1..9: note = (GSG)3 Linker
GSGGSGGSG

SEQ ID NO: 8 - protein, 12 aa, synthetic construct
  REGION 1..12: note = (GSG)4 Linker
GSGGSGGSGGSG

SEQ ID NO: 9 - protein, 4 aa, synthetic construct
  REGION 1..4: note = G3S Linker
GGGS

SEQ ID NO: 10 - protein, 8 aa, synthetic construct
  REGION 1..8: note = (G3S)2 Linker
GGGSGGGS

SEQ ID NO: 11 - protein, 12 aa, synthetic construct
  REGION 1..12: note = (G3S)3 Linker
GGGSGGGSGGGS

SEQ ID NO: 12 - protein, 16 aa, synthetic construct
  REGION 1..16: note = (G3S)4 Linker
GGGSGGGSGGGSGGGS

SEQ ID NO: 13 - protein, 5 aa, synthetic construct
  REGION 1..5: note = G4S Linker
GGGGS

SEQ ID NO: 14 - protein, 10 aa, synthetic construct
  REGION 1..10: note = (G4S)2 Linker
GGGGSGGGGS

SEQ ID NO: 15 - protein, 15 aa, synthetic construct
  REGION 1..15: note = (G4S)3 Linker
GGGGSGGGGSGGGGS

SEQ ID NO: 16 - protein, 20 aa, synthetic construct
  REGION 1..20: note = (G4S)4 Linker
GGGGSGGGGSGGGGSGGGGS

SEQ ID NO: 17 - protein, 4 aa, synthetic construct
  REGION 1..4: note = SGSG Linker
SGSG

SEQ ID NO: 18 - protein, 7 aa, synthetic construct
  REGION 1..7: note = S(GSG)2 Linker
SGSGGSG

SEQ ID NO: 19 - protein, 10 aa, synthetic construct
  REGION 1..10: note = S(GSG)3 Linker
SGSGGSGGSG

SEQ ID NO: 20 - protein, 13 aa, synthetic construct
  REGION 1..13: note = S(GSG)4 Linker
SGSGGSGGSGGSG

SEQ ID NO: 21 - protein, 5 aa, synthetic construct
  REGION 1..5: note = SG3S Linker
SGGGS

SEQ ID NO: 22 - protein, 9 aa, synthetic construct
  REGION 1..9: note = S(G3S)2 Linker
SGGGSGGGS

SEQ ID NO: 23 - protein, 13 aa, synthetic construct
  REGION 1..13: note = S(G3S)3 Linker
SGGGSGGGSGGGS

SEQ ID NO: 24 - protein, 17 aa, synthetic construct
  REGION 1..17: note = S(G3S)4 Linker
SGGGSGGGSGGGSGGGS

SEQ ID NO: 25 - protein, 6 aa, synthetic construct
  REGION 1..6: note = SG4S Linker
SGGGGS

SEQ ID NO: 26 - protein, 11 aa, synthetic construct
  REGION 1..11: note = S(G4S)2 Linker
SGGGGSGGGGS

SEQ ID NO: 27 - protein, 16 aa, synthetic construct
  REGION 1..16: note = S(G4S)3 Linker
SGGGGSGGGGSGGGGS

SEQ ID NO: 28 - protein, 21 aa, synthetic construct
  REGION 1..21: note = S(G4S)4 Linker
SGGGGSGGGGSGGGGSGGGGS

SEQ ID NO: 29 - protein, 5 aa, synthetic construct
  REGION 1..5: note = EAAAK Linker
EAAAK

SEQ ID NO: 30 - protein, 10 aa, synthetic construct
  REGION 1..10: note = (EAAAK)2 Linker
EAAAKEAAAK

SEQ ID NO: 31 - protein, 15 aa, synthetic construct
  REGION 1..15: note = (EAAAK)3 Linker
EAAAKEAAAKEAAAK

SEQ ID NO: 32 - protein, 20 aa, synthetic construct
  REGION 1..20: note = (EAAAK)4 Linker
EAAAKEAAAKEAAAKEAAAK

SEQ ID NO: 33 - protein, 5 aa, synthetic construct
  REGION 1..5: note = PAPAP Linker
PAPAP

SEQ ID NO: 34 - protein, 10 aa, synthetic construct
  REGION 1..10: note = (PAPAP)2 Linker
PAPAPPAPAP

SEQ ID NO: 35 - protein, 15 aa, synthetic construct
  REGION 1..15: note = (PAPAP)3 Linker
PAPAPPAPAPPAPAP

SEQ ID NO: 36 - protein, 20 aa, synthetic construct
  REGION 1..20: note = (PAPAP)4 Linker
PAPAPPAPAPPAPAPPAPAP

SEQ ID NO: 37 - protein, 8 aa, synthetic construct
  REGION 1..8: note = VLVH Linker
IKRTVAAP

SEQ ID NO: 38 - protein, 5 aa, synthetic construct
  REGION 1..5: note = RAKPS Linker
RAKPS

SEQ ID NO: 39 - protein, 10 aa, synthetic construct
  REGION 1..10: note = (RAKPS)2 Linker
RAKPSRAKPS

SEQ ID NO: 40 - protein, 5 aa, synthetic construct
  REGION 1..5: note = ASTKG Linker
ASTKG

SEQ ID NO: 41 - protein, 10 aa, synthetic construct
  REGION 1..10: note = (ASTKG)2 Linker
ASTKGASTKG

SEQ ID NO: 42 - protein, 5 aa, synthetic construct
  REGION 1..5: note = AKTHT Linker
AKTHT

SEQ ID NO: 43 - protein, 10 aa, synthetic construct
  REGION 1..10: note = (AKTHT)2 Linker
AKTHTAKTHT

SEQ ID NO: 44 - protein, 15 aa, synthetic construct
  REGION 1..15: note = ASTKG(G4S)2 Linker
ASTKGGGGGSGGGGS

SEQ ID NO: 45 - protein, 15 aa, synthetic construct
  REGION 1..15: note = RAKPS(G4S)2 Linker
RAKPSGGGGSGGGGS

SEQ ID NO: 46 - protein, 133 aa, synthetic construct [sequence omitted]

SEQ ID NO: 47 - protein, 133 aa, synthetic construct
  REGION 1..133: note = hIL-2 (T3A)
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 48 - protein, 133 aa, synthetic construct
  REGION 1..133: note = hIL-2 (C125S)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 49 - protein, 133 aa, synthetic construct
  REGION 1..133: note = hIL-2 (C125A)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 50 - protein, 133 aa, synthetic construct
  REGION 1..133: note = hIL-2 (T3A, C125S)
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 51 - protein, 133 aa, synthetic construct
  REGION 1..133: note = hIL-2 (T3A, C125A)
APASSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 52 - protein, 133 aa, synthetic construct
  REGION 1..133: note = hIL-2 Mutein (R38N, L40T, C125S)
APTSSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 53 - protein, 133 aa, synthetic construct
  REGION 1..133: note = hIL-2 Mutein (T3A, R38N, L40T, C125S)
APASSSTKKTQLQLEHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 54 - protein, 133 aa, synthetic construct
  REGION 1..133: note = hIL-2 Mutein (E15K, R38N, L40T, C125S)
APTSSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 55 - protein, 133 aa, synthetic construct
  REGION 1..133: note = hIL-2 Mutein (T3A, E15K, R38N, L40T, C125S)
APASSSTKKTQLQLKHLLLDLQMILNGINNYKNPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 56 - protein, 133 aa, synthetic construct
  REGION 1..133: note = hIL-2 Mutein (E15K, K32E, R38N, L40T, C125S)
APTSSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 57 - protein, 133 aa, synthetic construct
  REGION 1..133: note = hIL-2 Mutein (T3A, E15K, K32E, R38N, L40T, C125S)
APASSSTKKTQLQLKHLLLDLQMILNGINNYENPKLTNMTTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 58 - protein, 133 aa, synthetic construct
  REGION 1..133: note = hIL-2 Mutein (E15K, R38N, L40T, K76E, C125S)
APTSSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 59 - protein, 133 aa, synthetic construct
  REGION 1..133: note = hIL-2 Mutein (T3A, E15K, R38N, L40T, K76E, C125S)
APASSSTKKTQLQLKHLLLDLQMILNGINNYKNPKLTNMTTFKFYMPKKATELKHLQCLEEELKPLEEVLNLAQSENFHLRPRDLISNINVIVLELKGSETTFMCEYADETATIVEFLNRWITFSQSIISTLT

SEQ ID NO: 60 - protein, 133 aa, synthetic construct
  REGION 1..133: note = hIL-2 Mutein (K32E, R38E, F42A, C125S)
APTSSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 61 - protein, 133 aa, synthetic construct
  REGION 1..133: note = hIL-2 Mutein (T3A, K32E, R38E, F42A, C125S)
APASSSTKKTQLQLEHLLLDLQMILNGINNYENPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 62 - protein, 133 aa, synthetic construct
  REGION 1..133: note = hIL-2 Mutein (K32E, R38E, F42K, C125S)
APTSSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 63 - protein, 133 aa, synthetic construct
  REGION 1..133: note = hIL-2 Mutein (T3A, K32E, R38E, F42K, C125S)
APASSSTKKTQLQLEHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 64 - protein, 133 aa, synthetic construct
  REGION 1..133: note = hIL-2 Mutein (E15K, K32E, R38E, F42K, C125S)
APTSSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 65 - protein, 133 aa, synthetic construct
  REGION 1..133: note = hIL-2 Mutein (T3A, E15K, K32E, R38E, F42K, C125S)
APASSSTKKTQLQLKHLLLDLQMILNGINNYENPKLTEMLTKKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 66 - protein, 133 aa, synthetic construct
  REGION 1..133: note = hIL-2 Mutein (R38E, F42A, K76E, C125S)
APTSSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 67 - protein, 133 aa, synthetic construct
  REGION 1..133: note = hIL-2 Mutein (T3A, R38E, F42A, K76E, C125S)
APASSSTKKTQLQLEHLLLDLQMILNGINNYKNPKLTEMLTAKFYMPKKATELKHLQCLEEELKPLEEVLNLAQSENFHLRPRDLISNINVIVLELKGSETTFMCEYADETATIVEFLNRWITFSQSIISTLT

SEQ ID NO: 68 - protein, 133 aa, synthetic construct
  REGION 1..133: note = hIL-2 Mutein (R38E, F42K, K76E, C125S)
APTSSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 69 - protein, 133 aa, synthetic construct
  REGION 1..133: note = hIL-2 Mutein (T3A, R38E, F42K, K76E, C125S)
APASSSTKKTQLQLEHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 70 - protein, 133 aa, synthetic construct
  REGION 1..133: note = hIL-2 Mutein (E15K, R38E, F42K, K76E, C125S)
APTSSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 71 - protein, 133 aa, synthetic construct
  REGION 1..133: note = hIL-2 Mutein (T3A, E15K, R38E, F42K, K76E, C125S)
APASSSTKKTQLQLKHLLLDLQMILNGINNYKNPKLTEMLTKKFYMPKKATELKHLQCLEEELKPLEEVLNLAQSENFHLRPRDLISNINVIVLELKGSETTFMCEYADETATIVEFLNRWITFSQSIISTLT

SEQ ID NO: 72 - protein, 133 aa, synthetic construct
  REGION 1..133: note = hIL-2 Mutein (F42C, V69C, C125S)
APTSSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 73 - protein, 133 aa, synthetic construct
  REGION 1..133: note = hIL-2 Mutein (T3A, F42C, V69C, C125S)
APASSSTKKTQLQLEHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 74 - protein, 133 aa, synthetic construct
  REGION 1..133: note = hIL-2 Mutein (K32E, F42C, V69C, C125S)
APTSSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 75 - protein, 133 aa, synthetic construct
  REGION 1..133: note = hIL-2 Mutein (T3A, K32E, F42C, V69C, C125S)
APASSSTKKTQLQLEHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 76 - protein, 133 aa, synthetic construct
  REGION 1..133: note = hIL-2 Mutein (F42C, V69C, K76E, C125S)
APTSSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 77 - protein, 133 aa, synthetic construct
  REGION 1..133: note = hIL-2 Mutein (T3A, F42C, V69C, K76E, C125S)
APASSSTKKTQLQLEHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 78 - protein, 133 aa, synthetic construct
  REGION 1..133: note = hIL-2 Mutein (E15K, F42C, V69C, C125S)
APTSSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 79 - protein, 133 aa, synthetic construct
  REGION 1..133: note = hIL-2 Mutein (T3A, E15K, F42C, V69C, C125S)
APASSSTKKTQLQLKHLLLDLQMILNGINNYKNPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 80 - protein, 133 aa, synthetic construct
  REGION 1..133: note = hIL-2 Mutein (E15K, K32E, F42C, V69C, C125S)
APTSSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 81 - protein, 133 aa, synthetic construct
  REGION 1..133: note = hIL-2 Mutein (T3A, E15K, K32E, F42C, V69C, C125S)
APASSSTKKTQLQLKHLLLDLQMILNGINNYENPKLTRMLTCKFYMPKKATELKHLQCLEEELKPLEECLNLAQSKNFHLRPRDLISNINVIVLELKGSETTFMCEYADETATIVEFLNRWITFSQSIISTLT

SEQ ID NO: 82 - protein, 133 aa, synthetic construct
  REGION 1..133: note = hIL-2 Mutein (E15K, F42C, V69C, K76E, C125S)
APTSSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 83 - protein, 133 aa, synthetic construct
  REGION 1..133: note = hIL-2 Mutein (T3A, E15K, F42C, V69C, K76E, C125S)
APASSSTKKTQLQLKHLLLDLQMILNGINNYKNPKLTRMLTCKFYMPKKATELKHLQCLEEELKPLEECLNLAQSENFHLRPRDLISNINVIVLELKGSETTFMCEYADETATIVEFLNRWITFSQSIISTLT

SEQ ID NO: 84 - protein, 12 aa, synthetic construct
  REGION 1..12: note = hIL-2 Fragment (N29 to L40)
NNYKNPKLTRML

SEQ ID NO: 85 - protein, 8 aa, synthetic construct
  REGION 1..8: note = IL-15 Hinge Fragment-containing Peptide 1
QSMEIDAT

SEQ ID NO: 86 - protein, 8 aa, synthetic construct
  REGION 1..8: note = IL-15 Hinge Fragment-containing peptide 2
QSMHIDAT

SEQ ID NO: 87 - protein, 129 aa, synthetic construct
  REGION 1..129: note = IL-2-IL-15 Chimera 1 (C125S)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 88 - protein, 129 aa, synthetic construct
  REGION 1..129: note = IL-2-IL-15 Chimera 2 (T3A, C125S)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 89 - protein, 129 aa, synthetic construct
  REGION 1..129: note = IL-2-IL-15 Chimera 3 (E32H, C125S)
APTSSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 90 - protein, 129 aa, synthetic construct
  REGION 1..129: note = IL-2-IL-15 Chimera 4 (T3A, E32H, C125S)
APASSSTKKTQLQLEHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 91 - protein, 129 aa, synthetic construct
  REGION 1..129: note = IL-2-IL-15 Chimera 5 (E15K, C125S)
APTSSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 92 - protein, 129 aa, synthetic construct
  REGION 1..129: note = IL-2-IL-15 Chimera 6 (T3A, E15K, C125S)
APASSSTKKTQLQLKHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 93 - protein, 129 aa, synthetic construct
  REGION 1..129: note = IL-2-IL-15 Chimera 7 (E15K, E32H, C125S)
APTSSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 94 - protein, 129 aa, synthetic construct
  REGION 1..129: note = IL-2-IL-15 Chimera 8 (T3A, E15K, E32H, C125S)
APASSSTKKTQLQLKHLLLDLQMILNGIQSMHIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 95 - protein, 129 aa, synthetic construct
  REGION 1..129: note = IL-2-IL-15 Chimera 9 (E15K, E32H, S87D, C125S)
APTSSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 96 - protein, 129 aa, synthetic construct
  REGION 1..129: note = IL-2-IL-15 Chimera 10 (T3A, E15K, E32H, S87D, C125S)
APASSSTKKTQLQLKHLLLDLQMILNGIQSMHIDATTFKFYMPKKATELKHLQCLEEELKPLEEVLNLAQSKNFHLRPRDLIDNINVIVLELKGSETTFMCEYADETATIVEFLNRWITFSQSIISTLT

SEQ ID NO: 97 - protein, 129 aa, synthetic construct
  REGION 1..129: note = IL-2-IL-15 Chimera 11 (H16E, C125S)
APTSSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 98 - protein, 129 aa, synthetic construct
  REGION 1..129: note = IL-2-IL-15 Chimera 12 (T3A, H16E, C125S)
APASSSTKKTQLQLEELLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 99 - protein, 129 aa, synthetic construct
  REGION 1..129: note = IL-2-IL-15 Chimera 13 (H16F, C125S)
APTSSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 100 - protein, 129 aa, synthetic construct
  REGION 1..129: note = IL-2-IL-15 Chimera 14 (T3A, H16F, C125S)
APASSSTKKTQLQLEFLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 101 - protein, 129 aa, synthetic construct
  REGION 1..129: note = IL-2-IL-15 Chimera 15 (H16I, C125S)
APTSSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 102 - protein, 129 aa, synthetic construct
  REGION 1..129: note = IL-2-IL-15 Chimera 16 (T3A, H16I, C125S)
APASSSTKKTQLQLEILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 103 - protein, 129 aa, synthetic construct
  REGION 1..129: note = IL-2-IL-15 Chimera 17 (H16V, C125S)
APTSSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 104 - protein, 129 aa, synthetic construct
  REGION 1..129: note = IL-2-IL-15 Chimera 18 (T3A, H16V, C125S)
APASSSTKKTQLQLEVLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 105 - protein, 129 aa, synthetic construct
  REGION 1..129: note = IL-2-IL-15 Chimera 19 (D20T, C125S)
APTSSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 106 - protein, 129 aa, synthetic construct
  REGION 1..129: note = IL-2-IL-15 Chimera 20 (T3A, D20T, C125S)
APASSSTKKTQLQLEHLLLTLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 107 - protein, 129 aa, synthetic construct
  REGION 1..129: note = IL-2-IL-15 Chimera 21 (D20A, C125S)
APTSSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 108 - protein, 129 aa, synthetic construct
  REGION 1..129: note = IL-2-IL-15 Chimera 22 (T3A, D20A, C125S)
APASSSTKKTQLQLEHLLLALQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 109 - protein, 129 aa, synthetic construct
  REGION 1..129: note = IL-2-IL-15 Chimera 23 (D20E, C125S)
APTSSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 110 - protein, 129 aa, synthetic construct
  REGION 1..129: note = IL-2-IL-15 Chimera 24 (T3A, D20E, C125S)
APASSSTKKTQLQLEHLLLELQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 111 - protein, 129 aa, synthetic construct
  REGION 1..129: note = IL-2-IL-15 Chimera 25 (D20K, C125S)
APTSSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 112 - protein, 129 aa, synthetic construct
  REGION 1..129: note = IL-2-IL-15 Chimera 26 (T3A, D20K, C125S)
APASSSTKKTQLQLEHLLLKLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 113 - protein, 129 aa, synthetic construct
  REGION 1..129: note = IL-2-IL-15 Chimera 27 (N88A, C125S)
APTSSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 114 - protein, 129 aa, synthetic construct
  REGION 1..129: note = IL-2-IL-15 Chimera 28 (T3A, N88A, C125S)
APASSSTKKTQLQLEHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 115 - protein, 129 aa, synthetic construct
  REGION 1..129: note = IL-2-IL-15 Chimera 29 (I92A, C125S)
APTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 116 - protein, 129 aa, synthetic construct
  REGION 1..129: note = IL-2-IL-15 Chimera 30 (T3A, I92A, C125S)
APASSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 117 - protein, 129 aa, synthetic construct
  REGION 1..129: note = IL-2-IL-15 Chimera 31 (I92G, C125S)
APTSSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 118 - protein, 129 aa, synthetic construct
  REGION 1..129: note = IL-2-IL-15 Chimera 32 (T3A, I92G, C125S)
APASSSTKKTQLQLEHLLLDLQMILNGIQSMEIDATTFKFYMPKKATELKHLQCLEEELKPLEEVLNLAQSKNFHLRPRDLISNINVGVLELKGSETTFMCEYADETATIVEFLNRWITFSQSIISTLT

SEQ ID NO: 119 - protein, 129 aa, synthetic construct
  REGION 1..129: note = IL-2-IL-15 Chimera 33 (I92D, C125S)
APTSSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 120 - protein, 129 aa, synthetic construct
  REGION 1..129: note = IL-2-IL-15 Chimera 34 (T3A, I92D, C125S)
APASSSTKKTQLQLEHLLLDLQMILNGIQSMEIDATTFKFYMPKKATELKHLQCLEEELKPLEEVLNLAQSKNFHLRPRDLISNINVDVLELKGSETTFMCEYADETATIVEFLNRWITFSQSIISTLT

SEQ ID NO: 121 - protein, 129 aa, synthetic construct
  REGION 1..129: note = IL-2-IL-15 Chimera 35 (I92E, C125S)
APTSSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 122 - protein, 129 aa, synthetic construct
  REGION 1..129: note = IL-2-IL-15 Chimera 36 (T3A, I92E, C125S)
APASSSTKKTQLQLEHLLLDLQMILNGIQSMEIDATTFKFYMPKKATELKHLQCLEEELKPLEEVLNLAQSKNFHLRPRDLISNINVEVLELKGSETTFMCEYADETATIVEFLNRWITFSQSIISTLT

SEQ ID NO: 123 - protein, 129 aa, synthetic construct
  REGION 1..129: note = IL-2-IL-15 Chimera 37 (H16E, I92A, C125S)
APTSSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 124 - protein, 129 aa, synthetic construct
  REGION 1..129: note = IL-2-IL-15 Chimera 38 (T3A, H16E, I92A, C125S)
APASSSTKKTQLQLEELLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 125 - protein, 129 aa, synthetic construct
  REGION 1..129: note = IL-2-IL-15 Chimera 39 (E15K, I92A, C125S)
APTSSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 126 - protein, 129 aa, synthetic construct
  REGION 1..129: note = IL-2-IL-15 Chimera 40 (T3A, E15K, I92A, C125S)
APASSSTKKTQLQLKHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSQSIISTLT

SEQ ID NO: 127 - protein, 129 aa, synthetic construct
  REGION 1..129: note = IL-2-IL-15 Chimera 41 (E15K, H16I, C125S)
APTSSSTKKTQLQLKILLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 128 - protein, 129 aa, synthetic construct
  REGION 1..129: note = IL-2-IL-15 Chimera 42 (E15K, N88A, C125S)
APTSSSTKKTQLQLKHLLLDLQMILNGIQSMEIDATTFKFYMPKKATELKHLQCLEEELKPLEEVLNLAQSKNFHLRPRDLISAINVIVLELKGSETTFMCEYADETATIVEFLNRWITFSQSIISTLT

SEQ ID NO: 129 - protein, 129 aa, synthetic construct
  REGION 1..129: note = IL-2-IL-15 Chimera 43 (L18C)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 130 - protein, 129 aa, synthetic construct
  REGION 1..129: note = IL-2-IL-15 Chimera 44 (T3A, L18C)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 131 - protein, 129 aa, synthetic construct
  REGION 1..129: note = IL-2-IL-15 Chimera 45 (C125S, Q126E)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 132 - protein, 129 aa, synthetic construct
  REGION 1..129: note = IL-2-IL-15 Chimera 46 (T3A, C125S, Q126E)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 133 - protein, 129 aa, synthetic construct
  REGION 1..129: note = IL-2-IL-15 Chimera 47 (C125S, Q126K)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSKSIISTLT

SEQ ID NO: 134 - protein, 129 aa, synthetic construct
  REGION 1..129: note = IL-2-IL-15 Chimera 48 (T3A, C125S, Q126K)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSKSIISTLT

SEQ ID NO: 135 - protein, 129 aa, synthetic construct
  REGION 1..129: note = IL-2-IL-15 Chimera 49 (C125S, Q126R)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSRSIISTLT

SEQ ID NO: 136 - protein, 129 aa, synthetic construct
  REGION 1..129: note = IL-2-IL-15 Chimera 50 (T3A, C125S, Q126R)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSRSIISTLT

SEQ ID NO: 137 - protein, 129 aa, synthetic construct
  REGION 1..129: note = IL-2-IL-15 Chimera 51 (C125S, Q126S)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSSSIISTLT

SEQ ID NO: 138 - protein, 129 aa, synthetic construct
  REGION 1..129: note = IL-2-IL-15 Chimera 52 (T3A, C125S, Q126S)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSSSIISTLT

SEQ ID NO: 139 - protein, 129 aa, synthetic construct
  REGION 1..129: note = IL-2-IL-15 Chimera 53 (C125S, Q126T)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 140 - protein, 129 aa, synthetic construct
  REGION 1..129: note = IL-2-IL-15 Chimera 54 (T3A, C125S, Q126T)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIISTLT

SEQ ID NO: 141 - protein, 129 aa, synthetic construct
  REGION 1..129: note = IL-2-IL-15 Chimera 55 (C125S, S130R)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIIRTLT

SEQ ID NO: 142 - protein, 129 aa, synthetic construct
  REGION 1..129: note = IL-2-IL-15 Chimera 56 (T3A, C125S, S130R)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSQSIIRTLT

SEQ ID NO: 143 - protein, 129 aa, synthetic construct
  REGION 1..129: note = IL-2-IL-15 Chimera 57 (L18C, Q126E)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 144 - protein, 129 aa, synthetic construct
  REGION 1..129: note = IL-2-IL-15 Chimera 58 (T3A, L18C, Q126E)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCESIISTLT

SEQ ID NO: 145 - protein, 129 aa, synthetic construct
  REGION 1..129: note = IL-2-IL-15 Chimera 59 (L18C, Q126K)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 146 - protein, 129 aa, synthetic construct
  REGION 1..129: note = IL-2-IL-15 Chimera 60 (T3A, L18C, Q126K)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCKSIISTLT

SEQ ID NO: 147 - protein, 129 aa, synthetic construct
  REGION 1..129: note = IL-2-IL-15 Chimera 61 (L18C, Q126R)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCRSIISTLT

SEQ ID NO: 148 - protein, 129 aa, synthetic construct
  REGION 1..129: note = IL-2-IL-15 Chimera 62 (T3A, L18C, Q126R)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCRSIISTLT

SEQ ID NO: 149 - protein, 129 aa, synthetic construct
  REGION 1..129: note = IL-2-IL-15 Chimera 63 (L18C, Q126S)
APTSSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 150 - protein, 129 aa, synthetic construct
  REGION 1..129: note = IL-2-IL-15 Chimera 64 (T3A, L18C, Q126S)
APASSSTKKTQLQLEHLCLDLQMILNGIQSMEIDATTFKFYMPKKATELKHLQCLEEELKPLEEVLNLAQSKNFHLRPRDLISNINVIVLELKGSETTFMCEYADETATIVEFLNRWITFCSSIISTLT

SEQ ID NO: 151 - protein, 129 aa, synthetic construct
  REGION 1..129: note = IL-2-IL-15 Chimera 65 (I92A, C125S, Q126E)
APTSSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSESIISTLT

SEQ ID NO: 152 - protein, 129 aa, synthetic construct
  REGION 1..129: note = IL-2-IL-15 Chimera 66 (T3A, I92A, C125S, Q126E)
APASSSTKKTQLQLEHLLLDLQMILNGIQSMEIDATTFKFYMPKKATELKHLQCLEEELKPLEEVLNLAQSKNFHLRPRDLISNINVAVLELKGSETTFMCEYADETATIVEFLNRWITFSESIISTLT

SEQ ID NO: 153 - protein, 129 aa, synthetic construct
  REGION 1..129: note = IL-2-IL-15 Chimera 67 (C125S, Q126T, S130R)
APTSSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIIRTLT

SEQ ID NO: 154 - protein, 129 aa, synthetic construct
  REGION 1..129: note = IL-2-IL-15 Chimera 68 (T3A, C125S, Q126T, S130R)
APASSSTKKTQLQLEHLLLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSTSIIRTLT

SEQ ID NO: 155 - protein, 129 aa, synthetic construct
  REGION 1..129: note = IL-2-IL-15 Chimera 69 (T3A, L18S, C125S, Q126E)
APASSSTKKTQLQLEHLSLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 156 - protein, 145 aa, synthetic construct
  REGION 1..145: note = Mouse IL-2-IL-15 Chimera
APTSSSTSSSTAEAQQQQQQQQQQQQHLEQLLMDLQELLSRMQSMEIDATTFKFYLPKQATELKDLQCLEDELGPLRHVLDLTQSKSFQLEDAENFISNIRVTVVKLKGSDNTFECQFDDESATVVDFLRRWIAFSQSIISTSPQ

SEQ ID NO: 157 - protein, 219 aa, synthetic construct
  REGION 1..219: note = hIL-2R (extracellular domain)
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 158 - protein, 213 aa, synthetic construct
  REGION 1..213: note = hIL-2R (extracellular domain)
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKD

SEQ ID NO: 159 - protein, 240 aa, synthetic construct
  REGION 1..240: note = hIL-2R (extracellular domain)
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 160 - protein, 490 aa, synthetic construct
  REGION 1..490: note = Anti-hPD-1- IL-2-IL-15 Chimera 69 (T3A, L18S, C125S, Q126E) (Hole)
QVQLLESGGGLVKPGGSLRLSCAASGRTYAMGWFRRAPGKESEFVAEIRWSSTFTDYADSVKGRFTISRDNAENSAYLQMNSLRAEDTAVYHCAAQTSQRGTLYDSYGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGAGGGGSGGGGSGGGGSAPASSSTKKTQLQLEHLSLDLQMILNGIQSMEIDATTFKFYMPKKATELKHLQCLEEELKPLEEVLNLAQSKNFHLRPRDLISNINVIVLELKGSETTFMCEYADETATIVEFLNRWITFSESIISTLT

SEQ ID NO: 161 - protein, 346 aa, synthetic construct
  REGION 1..346: note = Anti-hPD-1 (Knob)
QVQLLESGGGLVKPGGSLRLSCAASGRTYAMGWFRRAPGKESEFVAEIRWSSTFTDYADSVKGRFTISRDNAENSAYLQMNSLRAEDTAVYHCAAQTSQRGTLYDSYGQGTQVTVSSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 162 - protein, 227 aa, synthetic construct
  REGION 1..227: note = hIgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 163 - protein, 227 aa, synthetic construct
  REGION 1..227: note = hIgG1 Fc Mutein (N297A)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 164 - protein, 227 aa, synthetic construct
  REGION 1..227: note = hIgG1 Fc Mutein (LALA, N297A)
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 227 aa, synthetic construct
  REGION 1..227: note = hIgG1 Fc Mutein (S354C, T366W)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 166 - protein, 227 aa, synthetic construct
  REGION 1..227: note = hIgG1 Fc Mutein (Y349C, T366S, L368A, Y407V)
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 167 - protein, 227 aa, synthetic construct
  REGION 1..227: note = hIgG1 Fc Mutein (S354C, T366W, N297A)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 168 - protein, 227 aa, synthetic construct
  REGION 1..227: note = hIgG1 Fc Mutein (Y349C, T366S, L368A, Y407V, N297A)
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 169 - protein, 227 aa, synthetic construct
  REGION 1..227: note = hIgG1 Fc Mutein (S354C, T366W, LALA, N297A)
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 227 aa, synthetic construct
  REGION 1..227: note = hIgG1 Fc Mutein (Y349C, T366S, L368A, Y407V, LALA, N297A)
DKTHTCPPCPAPEAAGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 171 - protein, 228 aa, synthetic construct
  REGION 1..228: note = hIgG2 Fc
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 172 - protein, 229 aa, synthetic construct
  REGION 1..229: note = hIgG4 Fc
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 229 aa, synthetic construct
  REGION 1..229: note = hIgG4 Fc (Hole)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGA

SEQ ID NO: 174 - protein, 229 aa, synthetic construct
  REGION 1..229: note = hIgG4 Fc (Knob)
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 175 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK